(19) United States
(12) Patent Application Publication
(10) Pub. No.: US 2009/0158455 A1
(43) Pub. Date: Jun. 18, 2009

(54) COMPOSITIONS AND METHODS FOR MODULATING LYSINE PRODUCTION

(76) Inventors: Charles Gilvarg, Princeton, NJ (US); Thomas Leustek, Roselle PK., NJ (US); Andre Hudson, North Breswell, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/917,864

(22) PCT Filed: Jun. 16, 2006
(86) PCT No.: PCT/US2006/023582

§ 371 Date: Jul. 25, 2008

Related U.S. Application Data
(60) Provisional application No. 60/691,106, filed on Jun. 16, 2005.
(60) Provisional application No. 60/739,308, filed on Nov. 23, 2005.

(51) Int. Cl. A01H 5/00; C12N 15/54; C12N 15/81; C12N 1/21; C12N 5/04; C12N 9/10; C12N 15/74; C12Q 1/68; C12N 15/82; C12Q 1/48; C12N 5/10; C12N 1/13

(52) U.S. Cl. 800/278; 536/23.2; 435/320.1; 435/252.3; 435/419; 435/257.2; 800/298; 435/15; 435/410; 435/412; 435/414; 435/252.1; 435/468; 435/193; 435/6

(57) ABSTRACT

Compositions and methods which modulate LL-diaminopimelate aminotransferase are disclosed. Also provided are compositions and methods for enhancing lysine biosynthesis in a cell.

This application claims priority to U.S. Provisional Applications, 60/691,106 and 60/739,308 filed Jun. 16, 2005 and Nov. 23, 2005 respectively. The subject matter of each these applications is incorporated herein by reference.

Pursuant to 35 U.S.C §202 (c), it is acknowledged that the U.S. Government has certain rights in this invention, which was made in part with funds from the National Institutes of Health and the National Science Foundation, Grant Numbers IBN-0449542, GM069264, and GM55145.

### FIELD OF THE INVENTION

This invention relates to the field of amino acid biochemistry in plants and other organisms. More specifically, compositions and methods for modulating lysine biosynthesis are provided.

### BACKGROUND OF THE INVENTION

Several literature references and patent documents are cited throughout the present specification in order to better describe the state of the art to which the invention pertains. Each of these citations is incorporated by reference herein as though set forth in full.

Lysine biosynthesis in plants is known to occur by way of a pathway that utilizes the intermediate diaminopimelic acid (DAP; Vogel, 1959). However, the exact pathway used by plants is uncertain despite the propagation in recent reviews of the idea that it is identical to the DAP pathway in prokaryotes (Matthews, 1999; Velasco et al., 2002; Azevedo, 2003). In fact, three variants of the DAP pathway are known in prokaryotes (See FIG. 1) and it was unclear which, if any of them, occurs in plants. The prokaryotic pathways are mechanistically alike in that all of them produce tetrahydrodipicolinate (THDPA) from Asp semialdehyde through the sequential action of dihydrodipicolinate synthase (DapA) and dihydrodipicolinate reductase (DapB), and all carry out the same final reaction catalyzed by meso-diaminopimelate (m-DAP) decarboxylase (LysA). The differences between them lie in the reactions at the center of the pathway. The first type to have been discovered and the one that shows the widest taxonomic distribution uses N-succinylated intermediates (Gilvarg, 1959, 1961; Velasco et al., 2002). THDPA is succinylated by a succinylCoA-dependent transferase (DapD) that results in opening of the ring and exposure of a keto group that serves as the acceptor site for the next reaction, a Glu-dependent transamination. At least two different gene products, DapC and ArgD, have been shown to catalyze the transamination (Ledwidge and Blanchard, 1999; Fuchs et al., 2000; Cox and Wang, 2001; Hartmann et al., 2003). After aminotransfer, the succinyl group is removed by a desuccinylase (DapE) to form LL-diaminopimelate (LL-DAP; Wehrmann et al., 1994). An epimerase (DapF) then converts LL-DAP to m-DAP (Richaud et al., 1987). A second, less widely distributed pathway exists that is mechanistically identical to the N-succinylated pathway, but differs in that the intermediates are acetylated (Sundharadas and Gilvarg, 1967; Weinberger and Gilvarg, 1970). A third variant of the DAP pathway, which shows a very narrow taxonomic distribution, utilizes m-DAP dehydrogenase (Ddh) to convert THDPA to m-DAP, bypassing the use of acyl intermediates and the epimerase, shortening the central part of the pathway from four steps to one (Misono et al., 1976; White, 1983). None of the variants of the DAP pathway are found in most fungi, which synthesize Lys via an unrelated pathway using the intermediate α-aminoadipic acid (Velasco et al., 2002).

A recent analysis of the Arabidopsis (Arabidopsis thaliana) genome for orthologs of bacterial Lys biosynthesis genes revealed that DapD and Ddh could not be detected in this species even though functional DapA, DapB, DapF, and LysA orthologs were identified (Hudson et al., 2005). Although orthologs of DapC, ArgD, and DapE could be identified, for a variety of reasons, none of them were considered likely to function in Lys biosynthesis. In addition, the fact that Ddh, DapC, and DapE activities could not be detected in extracts from a variety of plant species (Chatterjee et al., 1994; Hudson et al., 2005) suggested that plants might use an alternative mechanism to bridge the metabolic gap between THDPA and LL-DAP. The simplest way to visualize such a conversion would involve direct transamination of the acyclic form of THDPA, L-2-amino-6-oxopimelate. Since the chemical equilibrium favors the cyclic form (Chrystal et al., 1995; Caplan et al., 2001), the aminotransferase activity would potentially be more easily detected in the reverse of the biosynthetic direction using O-aminobenzaldehyde (OAB), a compound that would form a colored dihydroquinazolinium adduct with THDPA (Schöpf and Steuer, 1947).

### SUMMARY OF THE INVENTION

The discovery of enzymes having aminotransferase activity which contribute to lysine biosynthesis is disclosed herein. In one aspect of the invention, a nucleic acid molecule encoding LL-DAP amino transferase is provided. An exemplary nucleic acid may be isolated any species shown in FIG. 10. Alternatively, the nucleic acid may be isolated from a crop plant. Vectors encoding the nucleic acids described above are also encompassed by the present invention. Also included are cells comprising such vectors and the enzymes produced by expression of the same. Such vectors may also include regulatory sequences to promote expression of the encoded AT in the plant plastid. Such sequences are well known to the skilled artisan. In cases, where the cell is a plant cell, plants regenerated from such cells also comprise an aspect of the invention. Overexpression of the LL-DAP-AT of the invention in plant cells should result in increased lysine content in a plant regenerated from said plant cell. Additionally, overexpresion of LL-DAP-AT in bacterial cells will increase the lysine content therein.

In yet another embodiment, methods for identifying compounds which modulate LL-diaminopimelate aminotransferase activity are provided. An exemplary method entails incubating the aminotransferase in the presence and absence of the compound being assayed under conditions which promote aminotransferase activity. The amount of catalysis observed (i.e., the interconversion of tetrahydrodipicolinate and LL-diaminopimelate) in the presence and absence of said test compound is determined, thereby identifying modulators of LL-diaminopimelate aminotransferase activity. Such compounds may either reduce or augment aminotransferase activity. The invention also encompasses performance of the foregoing method in vitro and in vivo. In cases where the method is performed in vitro, the isolated enzyme and the necessary substrates are incubated under conditions which are suitable to promote aminotransferase activity.

Another aspect of the invention includes test compounds identified by the method described above. Such compounds can include without limitation, herbicides, algaecides, antibiotic and antibacterial agents.

In yet another embodiment of the invention, an alternative method of enhancing the conversion of tetrahydrodipicolinate to L,L-diaminopimelate in a cell is provided. An exemplary method entails introducing a heterologous nucleic acid encoding DAP dehydrogenase into a plant cell. Suitable enzymes for this purpose include, without limitation, DAP dehydrogenase from Corynebacterium glutamicum shown in FIG. 10A. FIG. 10B provides the DAP dehydrogenase sequence from Bacillus sphaericus. Such plant cells may optionally comprise nucleic acids encoding the LL-DAP AT described herein. Additionally, plants regenerated from such plant cells are encompassed by the invention. In a preferred embodiment, such plants are crop plants.

Alternatively, the conversion of tetrahydrodipicolinate to LL-diaminopimelate in a cell may be enhanced by introducing a plurality of nucleic acids encoding the enzymes in DAP acyl transferase pathway into a plant cell. These enzymes include L-2,3,4,5-tetrahydrodipicolinate acyl-transferase, N-succinyl-L-diaminopimelic glutamic transaminase, and N-succinyl-L-alpha,epsilon-diaminopimelic acid deacylase, commonly referred to as DapD, DapC, and DapE, respectively. They are also identified by the Enzyme Commission nomenclature EC 2.3.1.117, EC 2.6.1.17 and EC 3.5.1.18, respectively. Representative amino acid sequences for these enzymes are provided in FIG. 11. Alternatively the acetylating DapD, DapC and Dap E enzymes may be employed. Provision of heterologous nucleic acids encoding these enzymes should also effectively increase the lysine content in a plant cell. Plant cells containing nucleic acids expressing the foregoing enzymes may also comprise a nucleic acid encoding the LL-DAP-AT described herein. As above, transgenic plants regenerated from such plant cells also comprise an aspect of the present invention.

### BRIEF DESCRIPTION OF THE DRAWINGS

FIG. 1. The mechanisms for DAP/Lys synthesis. The pathways labeled in the diagram include two variants that use either succinylCoA or acetylCoA. Another uses Ddh to directly convert THDPA to m-DAP. The enzyme described herein, LL-DAP-AT, directly converts THDPA to LL-DAP. The DAP dehydrogenase and LL-DAP-AT diagrams show only the enzymatic step that differentiates these pathways from the acyl-DAP pathways. Acronyms in the diagram include THDPA, L-2,3,4,5-tetrahydrodipicolinate; LL-DAP, LL-2,6-diaminopimelate; m-DAP, m-2,6-diaminopimelate; DapA, dihydrodipicolninate synthase; DapB, dihydrodipicolinate reductase; DapD, THDPA acyltransferase; DapC, N-acyl-L-2-amino-6-oxopimelate aminotransferase; DapE, N-acyl-LL-2,6-diaminopimelate deacylase; DapF, DAP epimerase; and LysA, m-DAP decarboxylase. The structures of the intermediates are shown on the left.

FIGS. 2A and 2B. LL-DAP-AT activity in Arabidopsis. FIG. 2A shows the time course of a complete reaction with 500 μg protein from Arabidopsis leaf extract (black circles), compared with a reaction lacking LL-DAP (white circles). FIG. 2B shows the relationship between reaction rate and the amount of protein added to the reaction. The 1-mL assay contained 100 μmol HEPESKOH (pH 7.6), 0.5 μmol amino donor, 2.0 μmol 2-OG, and 1.25 mg OAB. The reaction was incubated at 30° C. The protein extract was prepared from Arabidopsis leaves by grinding in liquid nitrogen with 100 mM HEPESKOH (pH 7.6), centrifugation at 10,000 g for 15 min, and buffer exchange using an Amicon Ultra 30,000 MWCO filter.

FIG. 3. Recombinant expression and purification of LL-DAP-AT. E. coli strain BL21-Codon Plus-RIPL carrying pET30b-AtDAT was grown and expression from the plasmid induced as described below. The gel shows the profile of 10 μg soluble proteins in uninduced cells compared to induced cells. Also shown is 0.5 μg overexpressed LL-DAP-AT purified by nickel-affinity chromatography. The SDS-PAGE gel contained 12.5% (w/v) acrylamide and was stained with Coomassie Blue.

FIG. 4. LL-DAP synthesis activity. The forward assay was conducted in two steps. In the first, THDPA was synthesized from m-DAP using CgDdh. The aminotransferase was assayed in the second step. The prereaction contained in 1 mL 100 μmol HEPESKOH (pH 7.5), 0.5 μmol NADP⁺, 0.5 μmol (black circle) or 0.05 μmol (black square) m-DAP, 0.3 mmol thio-NAD⁺, 0.3 μmol CoA, 0.5 μmol Glu, and 32 μg Ddh. The reaction was incubated at 22° C. and the increase in A₃₄₀ was recorded as a measure of m-DAP to THDPA conversion. Then 200 μg of 2-OG dehydrogenase (0.625 μmol min⁻¹mg⁻¹ protein) was added to the reactions with 0.5 μmol (gray circle) or 0.05 μmol (gray square) m-DAP followed by pure LL-DAP-AT, and the increase in A₃₉₈ was measured to calculate the activity of the aminotransferase.

FIG. 5. Complementation of dap mutants with At4g33680. A, Strains AT980 (dapD), AT984 (dapE), and AOH1 (dapD/dapE) were transformed with either the plasmid vector (pBAD33) or with the At4g33680 expression plasmid (pBAD33-AtDAT). Colonies were selected on LB medium with 50 μg mL⁻¹ DAP and 34 μg mL⁻¹ chloramphenicol. Individual colonies were then replica plated onto NZY medium supplemented with 0.2% (w/v) Ara without or with 50 μg mL⁻¹ DAP. The cultures were grown at 30° C. for 48 h. B, Diagram of the DAP pathway in E. coli with the reaction catalyzed by LL-DAP-AT indicated. The structure on the left is of THDPA and on the right of LL-DAP.

FIG. 6. Phylogenetic tree showing the relationship between LL-DAP-AT orthologs and DapC and ArgD orthologs. The protein sequences were aligned using ClustalW and the neighbor-joining tree was constructed using the program MEGA2 version 2.1 (Kumar et al., 2001). The GenBank accession number or locus tag for the protein sequences used to produce the alignment were Avar03004417, At4g33680, NP_{—}389004.1, NP_{—}882054.1, BPP1996, AAO12273.1, Cwat03005178, CMN323C, Q8X4S6, AAU93923.1, Npun02008059, AY338235.1, Pro-1655, syc0687, SYNW2147, sll0480, tll2102, and Tery02000376. The protein from Thalassiosira pseudonana was deduced from gene model grail.111.8.1 on scaffold_{—}111 obtained from the Web site http://genome.jgi-psf.org/thaps1/thaps1.home.html. The protein from Medicago truncatula was deduced from the sequence between nucleotides 105403 and 110280 of bac clone mth2-36a23 (GenBank accession AC124214). The protein from Physcomitrella patens was deduced from contigs 10435 and 10436 obtained from http://moss.nibb.ac.jp/.

FIG. 7. Genomic context of DAP aminotransferase in various microbial species. The genomic context of the DAP aminotransferase gene (dapL) in various microbial species is shown. The dapL gene is in red. Nearby genes, suggestive of an operon structure with dapL are labelled.

FIG. 8. Phylogenetic tree showing the relationship between LL-DAP-AT orthologs and DapC and ArgD orthologs. The protein sequences were aligned using ClustalW and the neighbor-joining tree was constructed using the program MEGA2 version 2.1 (Kumar et al., 2001). Green dots indicate those enzymes that have been experimentally determined to catalyze LL-DAP aminotransferase activity. The locus tag numbers associated with each DapL sequence can be found in FIG. 10.

FIG. 9. A table showing the “best hit” orthologs of At4g33680 in the microbial genomes proteins database.

FIGS. 10A and 10B. The amino acid sequences of DAP enzymes useful to enhance lysine biosynthesis in higher plants.

FIG. 11. The amino acid sequences of enzymes useful for expressing the DAP acyl transferase pathway, DapD, DapC, and DapE are shown.

### DETAILED DESCRIPTION OF THE INVENTION

Although lysine (Lys) biosynthesis in plants is known to occur by way of a pathway that utilizes diaminopimelic acid (DAP) as a central intermediate, the available evidence suggests that none of the known DAP-pathway variants found in nature occur in plants. A new Lys biosynthesis pathway has been identified in Arabidopsis (Arabidopsis thaliana) that utilizes a novel transaminase that specifically catalyzes the interconversion of tetrahydrodipicolinate and LL-diaminopimelate, a reaction requiring three enzymes in the DAP-pathway variant found in Escherichia coli. The LL-DAP aminotransferase encoded by locus At4g33680 was able to complement the dapD and dapE mutants of E. coli. This result, in conjunction with the kinetic properties and substrate specificity of the enzyme, indicated that LL-DAP aminotransferase functions in the Lys biosynthetic direction under in vivo conditions. Orthologs of At4g33680 were identified in all the cyanobacterial species whose genomes have been sequenced. The Synechocystis sp. ortholog encoded by locus sll0480 showed the same functional properties as At4g33680. These results demonstrate that the Lys biosynthesis pathway in plants and cyanobacteria is distinct from the pathways that have so far been defined in microorganisms.

The synthesis of meso-diaminopimelic acid (m-DAP) in bacteria is essential for both peptidoglycan and lysine biosynthesis. From genome sequencing data, it was unclear how bacteria of the Chlamydiales order would synthesize m-DAP in the absence of dapD, dapC and dapE, which are missing from the genome. Here, we assessed the biochemical capacity of Chlamydia trachomatis serovar L2 to synthesize m-DAP. Screening of C. trachomatis genomic DNA for proteins having similarity to that encoded by At4g33680 revealed ct390 as encoding an enzyme that possessed aminotransferase activity. This hypothesis was supported by in vitro kinetic analysis of the CT390 protein and the fact that similar properties were demonstrated for the Protochlamydia amoebophila homologue pc0685.

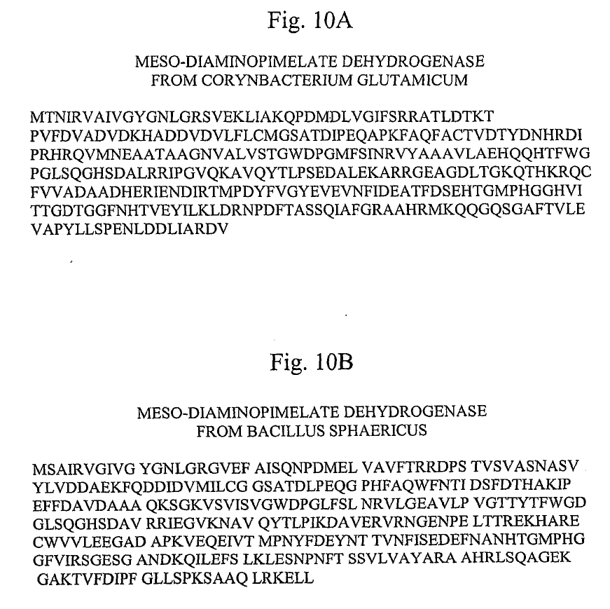

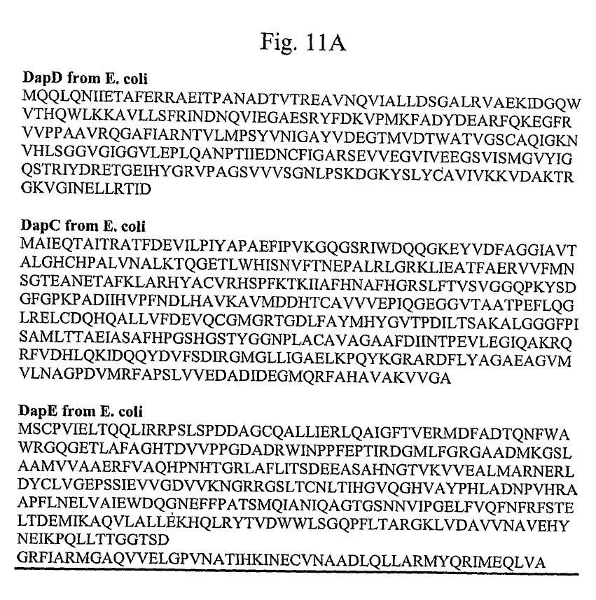

In yet another approach for enhancing lysine content in a cell, particularly a plant cell, transgenic plants are provided which overexpress DAP dehydrogenase. Overexpression of DAP dehydrogenase should make greater amounts of m-DAP available which could then be converted to lysine. An exemplary method entails introducing a heterologous nucleic acid encoding DAP dehydrogenase into a plant cell. Suitable enzymes for this purpose include, without limitation, DAP dehydrogenase from Corynebacterium glutamicum shown in FIG. 10A. FIG. 10B provides the DAP dehydrogenase sequence from Bacillus sphaericus. Such plant cells may optionally comprise nucleic acids encoding the LL-DAP AT described herein. Additionally, plants regenerated from such plant cells are encompassed by the invention. In a preferred embodiment, such plants are crop plants.

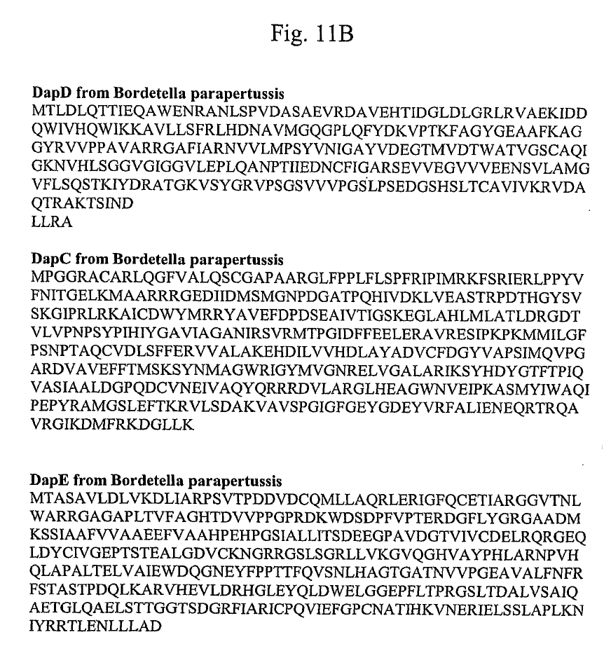

An alternative approach involves heterologous expression of the DAP acyl transferase pathway. Thus, nucleic acids encoding these enzymes, which include L-2,3,4,5-tetrahydrodipicolinate acyl-transferase, N-succinyl-L-diaminopimelic glutamic transaminase, and N-succinyl-L-alpha,epsilon-diaminopimelic acid deacylase, commonly referred to as DapD, DapC, and DapE, respectively can be introduced into plant cells. These enzymes are also identified by the Enzyme Commission nomenclature EC 2.3.1.117, EC 2.6.1.17 and EC 3.5.1.18, respectively. Representative amino acid sequences for the DapD, DapC, and DapE, enzymes are provided in FIG. 11. Alternatively the acetylating DapD, DapC and Dap E enzymes may be employed. Provision of these enzymes should also effectively increase the lysine content in a plant cell. Plant cells containing nucleic acids expressing the foregoing enzymes may also comprise the LL-DAP-AT described herein. As above, transgenic plants, preferably crop plants, regenerated from such plant cells also comprise an aspect of the present invention.

Lysine is important to humans on a number of counts. It is required for protein synthesis. The lysine biosynthesis pathway has been a prime target for discovery of antibiotics against pathogenic microorganisms since a part of the pathway is used for the synthesis of the peptidoglycan cell wall component. Lysine is also an essential nutrient for animals. The content of lysine limits the nutritional value of crop plants. Because of its importance in plant growth lysine biosynthesis is a prime target for development of antibiotics, agricultural herbicides, and algaecides.

All of the abovementioned areas are potential targets for commercial development. Improvement of the nutritional value of crops is currently a major goal for agricultural companies. Fermentative production of lysine for sale as nutritional supplement is a major industry. Antibiotics are also of major importance in both medicine, where they are used to counteract bacterial infections, and in agriculture or environmental applications, where they are used to eliminate weeds (herbicides) or algae (algaecides). Antibiotics, herbicides and algaecides together comprise major industries world-wide. Commercial exploitation of lysine biosynthesis depends on detailed knowledge of the biosynthesis pathway. Until the discovery that is presented herein, it was unclear exactly how lysine is synthesized by plants. Moreover, although the lysine biosynthesis pathway of certain bacteria was known, it was not obvious that other prokaryotic species have a different lysine biosynthesis pathway with greater similarity to the plant pathway. The genetic basis for lysine biosynthesis in plants is described herein. Moreover, the data presented herein indicate that a plant-like lysine biosynthesis pathway exists in some prokaryotic organisms including pathogens.

The following definitions are provided to facilitate an understanding of the present invention.

The term “LL-DAP-aminotransferase” refers to the enzyme which catalyzes the interconversion of tetrahydrodipicolinate and LL-diaminopimelate.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, may refer to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):

Tₘ=81.5C+16.6 Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, organ, or cell type, a promoter may display “enhanced” expression, i.e., a higher level of expression, in one part (e.g., cell type, tissue, or organ) of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

The 3′ non-translated region of coding regions of the nucleic acids of the invention typically contain a transcriptional terminator, or an element having equivalent function, and, optionally, a polyadenylation signal, which functions to cause the addition of polyadenylated nucleotides to the 3′ end of the RNA. Examples of suitable 3′ regions for use in plants are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of another 3′ region is that from the ssRUBISCO E9 gene from pea (European Patent Application 385,962, herein incorporated by reference in its entirety).

As used herein, “transgenic plant” includes reference to a plant that comprises within its nuclear genome a heterologous polynucleotide. Generally, the heterologous polynucleotide is stably integrated within the nuclear genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The phrase “crop plant” includes any plant cultivated for food or ornamentation with the exception of weeds. The crop plants for which lysine biosynthesis may be enhanced include, without limitation, corn, sugarcane, beans, rice, wheat, oats, soybean, tobacco, sorghum, and a wide variety of vegetables such as tomatoes, and fruits such as strawberries are examples. In a preferred embodiment, enzyme(s) which contribute to lysine biosynthesis are introduced into the following: (Zea mays), sorghum (Sorghum halepense), sorghum (Sorghum bicolor), soybean (Glycine max) or dry bean (Phaseoulus vulgaris L.). Examples of other cultivated plants for which lysine biosynthesis may be enhanced according to the present invention are herb plants such as parsley, sage, rosemary, and thyme.

The terms “percent similarity”, “percent identity” and “percent homology” when referring to a particular sequence are used as set forth in the University of Wisconsin GCG software program.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant. A number of “selectable marker genes” are known in the art and several antibiotic resistance markers satisfy these criteria, including those resistant to kanamycin (nptII), hygromycin B (aph IV) and gentamycin (aac3 and aacC4). Useful dominant selectable marker genes include genes encoding antibiotic resistance genes (e.g., resistance to hygromycin, kanamycin, bleomycin, G418, streptomycin or spectinomycin); and herbicide resistance genes (e.g., phosphinothricin acetyltransferase). A useful strategy for selection of transformants for herbicide resistance is described, e.g., in Vasil, Cell Culture and Somatic Cell Genetics of Plants, Vols. I III, Laboratory Procedures and Their Applications Academic Press, New York, 1984. Particularly preferred selectable marker genes for use in the present invention would genes which confer resistance to compounds such as antibiotics like kanamycin, and herbicides like glyphosate (Della-Cioppa et al., Bio/Technology 5(6), 1987, U.S. Pat. Nos. 5,463,175, 5,633,435). Other selection devices can also be implemented and would still fall within the scope of the present invention.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

“Native” refers to a naturally occurring (“wild-type”) nucleic acid sequence.

“Heterologous” sequence refers to a sequence which originates from a foreign source or species or, if from the same source, is modified from its original form.

A “coding sequence” or “coding region” refers to a nucleic acid molecule having sequence information necessary to produce a gene product, when the sequence is expressed.

“Genetic component” refers to any nucleic acid sequence or genetic element which may also be a component or part of an expression vector. Examples of genetic components include, but are not limited to promoter regions, 5′ untranslated leaders or promoters, introns, genes, 3′ untranslated regions or terminators, and other regulatory sequences or sequences which affect transcription or translation of one or more nucleic acid sequences.

“Complementary” refers to the natural association of nucleic acid sequences by base-pairing (A-G-T pairs with the complementary sequence T-C-A). Complementarity between two single-stranded molecules may be partial, if only some of the nucleic acids pair are complementary; or complete, if all bases pair are complementary. The degree of complementarity affects the efficiency and strength of hybridization and amplification reactions.

“Homology” refers to the level of similarity between nucleic acid or amino acid sequences in terms of percent nucleotide or amino acid positional identity, respectively, i.e., sequence similarity or identity. Homology also refers to the concept of similar functional properties among different nucleic acids or proteins.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

As used herein, an “instructional material” includes a publication, a recording, a diagram, or any other medium of expression which can be used to communicate the usefulness of the composition of the invention for performing a method of the invention. The instructional material of the kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

The examples set forth below are provided to illustrate certain embodiments of the invention. They are not intended to limit the invention in any way.

### Example I

### Identification of a Novel Lysine Biosynthesis Pathway in Higher Plants

The following materials and methods are provided to facilitate the practice of the present invention.

### Microbial Strains

The microbial strains used in this study are listed along with their contributors: Escherichia coli strains AT980, AT984, and AT999 (Coli Genetic Stock Center), JC7623 (Cranenburgh et al., 2001), BL21(DE3)/pET28-CgDDH expressing Corynebacterium glutamicum Ddh (D. I. Roper, University of Warwick), Synechocystis sp. PCC6803 and Bacteriophage P1kc (American Type Culture Collection, nos. 27184 and 25404-B1, respectively), Synechococcus sp. PCC7942 (B. Zilinskas, Rutgers University), Bacillus subtilis 168 (Bacillus Genetic Stock Center), Rhizobium tropici USDA9030 (U.S. Department of Agriculture-Agricultural Research Service National Rhizobium Germplasm Collection), and Agrobacterium tumefaciens GV3101 (Koncz and Schell, 1986). Molecular biology techniques were performed as generally described by Sambrook et al. (1989). E. coli strain AOH1 was constructed by transduction of ΔdapD::Kan2 from JC7623 into AT984 using P1kc. Replacement of dapD⁺ with dapD::Kan2 was confirmed by PCR using primers 5′-AATGGAGATCGGCCAGAAAAA-3′ (SEQ ID NO: 1) and 5′-GGTGCCCGAATTACAACCATT-3′ (SEQ ID NO: 2).

### Plants and Growth Conditions

Arabidopsis (Arabidopsis thaliana) Col7 (Arabidopsis Biological Resource Center), Glycine max, spinach (Spinacia oleracea), Brassica napus, and pea (Pisum sativum) Progress 9 were grown in peat-based PRO-MIX BX and fertilized with Peter's nutrients 20:20:20 (N:P:K) in a growth chamber with 16-h-light and 8-h-dark periods. The temperature was 24° C. during the light period and 20° C. during the dark. Light intensity was 120 μE m⁻²s⁻¹. Arabidopsis was also grown axenically in Murashige and Skoog liquid medium with minimal organics (Sigma-Aldrich product no. M6899). Surface-sterilized seed were sown into 50-mL medium in a 250-mL Erlenmeyer flask and were grown for 10 d with constant mixing on an orbital shaker at 50 rpm. Convallaria majalis was collected from the field. Maize (Zea mays) was from an embryogenic culture (Singh et al., 1988). Chlamydomonas reinhardtii and Physcomitrella patens were grown as described (Gorman and Levine, 1966; Schaefer et al., 1991).

cDNA Cloning and Protein Expression

The cDNA derived from At4g33680 was amplified by reverse transcription (RT)-PCR using the primers 5′-GGGGCATTGGAAGGAGATATAACCATGGCAGTCAATACTTGCAAATGT-3′ (SEQ ID NO: 3) and 5′-GGGGGTCGACTCATTTGTAAAGCTGCTTGAATCTTCG-3′ (SEQ ID NO: 4). Total RNA was isolated from 25-d-old Arabidopsis leaf using Trizol reagent (Life Technologies). RT was carried out with Superscript II RNAse H—Reverse Transcriptase system (Invitrogen, catalog no. 18064-014) using 1 μg of total RNA and an oligo(dT) primer. PCR was then carried out with the gene-specific primers using 12 pM of each primer, 1 mM MgSO₄, 0.5 mM of each of the four deoxynucleotide triphosphates, 2 μL RT reaction, and 1 unit of Platinum Pfx DNA polymerase using the following conditions: 1 cycle at 94° C., 2 min; and 36 cycles at 94° C. for 15 s, 60° C. for 30 s, and 72° C. for 2 min. The DNA fragment was digested with NcoI and SalI and cloned into pET30b to produce pET30-AtDAT. The recombinant protein lacks the first 39 amino acids of the At4g33680 protein and carries hexa-His and S-TAG sequence derived from pET30b at its amino terminus. Synechocystis sp. sll0480 was amplified from genomic DNA by PCR using the primers 5′-GGGGGGATCCATGGCCAGTATCAACGACAAC-3′ (SEQ ID NO: 5) and 5′-GGGGGTCGACCTAACCCAATTTGAGGGTGGA-3′ (SEQ ID NO: 6). The DNA fragment was digested with BamHI and SalI and cloned into pET30b to produce pET30-SsDAT. The recombinant protein derived from this plasmid carries the affinity tags fused to the amino terminus of the full-length sll0480 protein. pET30b-AtDAT and pET30b-SsDAT were transformed into E. coli BL21-CodonPlus-RIPL. Plasmids for functional complementation of E. coli dap mutants were produced by subcloning the XbaI and SalI fragment from pET30-AtDAT or pET30-SsDAT into pBAD33 (Guzman et al., 1995) to produce pBAD33-AtDAT and pBAD33-SsDAT. The fusion proteins produced from the pBAD33 constructs were identical to those from the pET30b constructs.

For protein expression and purification, the strains were grown on Luria-Bertani (LB) medium at 37° C. to an OD600 nm of 0.5 and protein expression was then induced with 1 mM isopropylthio-β-galactoside for 4 h at 25° C. Cells were lysed by sonication in a solution of 50 mM sodium phosphate and 300 mM NaCl (pH 8.0). The soluble fraction was incubated with Talon metal affinity agarose (CLONTECH no. 8901-2), washed three times with lysis buffer containing 10 mM imidazole, and eluted with 300 mM imidazole. The protein was concentrated in an Amicon Ultra 30,000 molecular weight cutoff (MWCO) ultrafilter, replacing the elution buffer with 100 mM HEPESKOH, pH 7.6. For the E. coli culture expressing C. glutamicum Ddh, the protein was not purified because it comprised approximately 90% of the soluble protein. The preparation converted m-DAP to THDPA at a rate of 14 μmol min⁻¹mg⁻¹ protein at 30° C.

### Functional Complementation and Enzyme Assays

In functional complementation dap mutant strains were transformed with either the plasmid vector or with LL-DAP-AT expression plasmids. Transformants were selected on LB medium supplemented with 50 μg mL⁻¹ DAP (DL_{-α,s-}DAP, Sigma-Aldrich product no. D-1377) and 34 μg mL⁻¹ chloramphenicol. Individual colonies were then replica plated onto NZY medium (5 g L⁻¹ NaCl, 2 g L⁻¹ MgSO₄-7H₂O, 10 g L⁻¹ caseine hydrolysate, 5 g L⁻¹ yeast extract, 15 g L⁻¹ agar) supplemented with 0.2% (w/v) Ara without or with 50 μg mL⁻¹ DAP. The cultures were grown at 30° C. for 48 h.

For enzyme assays of crude proteins, extracts were prepared by grinding tissue in liquid nitrogen with 100 mM HEPESKOH (pH 7.6), followed by centrifugation at 10,000 g for 15 min, and then buffer exchange using an Amicon Ultra 30,000 MWCO filter. The OAB assay contained in 1 mL 100 μmol HEPESKOH (pH7.6), 0.5 μmol amino donor, 2.0 μmol 2-OG, and 1.25 mg OAB, and crude soluble protein or pure protein. Reactions were incubated at 30° C. and the ΔA (440 nm) measured continuously. Quantitative assay of the physiologically reverse activity was measured in 1 mL containing 100 μmol HEPESKOH (pH 7.5), 0.3 μmol NADPH, 50 μmol NH₄Cl, 0.5 μmol LL-DAP, 5 μmol 2-OG, 16 μg CtDdh, and pure LL-DAP-AT. The reaction was incubated at 30° C., and the decrease in A₃₄₀ was measured. Quantitative assay of the physiologically forward reaction was measured in 1 mL containing 100 μmol HEPESKOH (pH 7.5), 0.5 μmol NADP⁺, varying concentrations of m-DAP, 0.3 μmol thio-NAD⁺, 0.3 μmol CoA, 0.5 μmol Glu, and 32 μg Ddh. The reaction was run to completion, determined by monitoring the increase in A₃₄₀. Then 200 μg of 2-OG dehydrogenase (0.625 μmol min⁻¹mg⁻¹ protein) and pure LL-DAP-AT were added. The kinetic constants were calculated by nonlinear regression analysis using GraphPad Prizm version 4.03.

The amino acid sequence and nucleic acid sequence encoding LL-DAP amino transferase from Arabidopsis are set forth below.

| Amino acid sequence (SEQ ID NO: 7) | |
|---|---|
| MSSTHQLVSSMISSSSSTFLAPSNFNLRTRNACLPMAKRVNTCKCVATPQEKIEYKTKVSRNSNMSKL | |
| QAGYLFPEIARRRSAHLLKYPDAQVISLGIGDTTEPIPEVITSAMAKKAHELSTIEGYSGYGAEQGAK | |
| PLRAAIAKTFYGGLGIGDDDVFVSDGAKCDISRLQVMFGSNVTIAVQDPSYPAYVDSSVIMGQTGQFN | |
| TDVQKYGNIEYMRCTPENGFFPDLSTVGRTDIIFFCSPNNPTGAAATREQLTQLVEFAKKNGSIIVYD | |
| SAYAMYMSDDNPRSIFEIPGAEEVAMETASFSKYAGFTGVRLGWTVIPKKLLYSDGFPVAKDFNRIIC | |
| TCFNGASNISQAGALACLTPEGLEAMHKVIGFYKENTNIIIDTFTSLGYDVYGGKNAPYVWVHFPNQS | |
| SWDVFAEILEKTHVVTTPGSGFGPGGEGFVRVSAFGHRENILEACRRFKQLYK | |
| Nucleic acid sequence (SEQ ID NO: 8): | |
| 1 | tttgctctgc aaatttgtct gaaaaaatct ttgtgcgaag caggaaaaat gtcgtcgacc |
| 61 | catcagttag tttcttcgat gatctcttct tcctcatcca ctttcttagc cccffcaaat |
| 121 | tffaatctca gaactcgaaa tgcffgctta cccatggcaa aacgggtcaa tacttgcaaa |
| 181 | tgtgttgcta cgccgcaaga gaagatcgag tataagacca aagtgtcacg gaattcaaac |
| 241 | atgtccaaac ttcaggctgg atacctattc ccggagattg caagaagaag gtctgcacac |
| 301 | ttgttgaaat atccagatgc acaagttata agtcffggaa taggcgacac aactgagcca |
| 361 | attcctgaag tgatcacttc tgctatggca aagaaagctc atgagttgtc aacaatagag |
| 421 | ggatatagtg gttatggtgc tgaacaaggt gcaaagccac tgagagctgc tattgcgaaa |
| 481 | acattctacg gtggccttgg cataggggat gatgacgttt ttgtttctga tggagctaaa |
| 541 | tgtgatatct cacgtctcca ggttatgttt ggttccaatg ttacaattgc tgttcaggat |
| 601 | ccttcatatc cggcttatgt ggactccagt gttattatgg gtcagactgg gcaatttaac |
| 661 | actgatgtgc aaaagtatgg aaacatcgag tacatgagat gcactccaga gaatggcttc |
| 721 | tttcccgact tgtccaccgt tggcaggaca gatataattt tcttctgttc cccaaataac |
| 781 | cctacgggtg ctgctgccac gagagagcaa ctaacgcagt tagffgagtt tgcaaagaag |
| 841 | aacggttcta taatagtgta tgattccgcc tatgcaatgt acatgtctga tgataaccca |
| 901 | cgatccatct tcgaaatccc tggagcagag gaggtcgcta tggagacagc ttcgttcagc |
| 961 | aaatatgctg gtttcactgg agttcgactt ggttggactg tcatcccgaa aaagctactc |
| 1021 | tattcagacg gtttccctgt tgccaaagac ttcaatcgga ttatctgcac ttgtttcaat |
| 1081 | ggtgcatcta atatctctca agctggtgct cttgcttgcc ttacacccga aggacttgag |
| 1141 | gcaatgcata aggtgattgg attctataaa gaaaacacaa acataatcat tgacacattc |
| 1201 | acatctctcg ggtatgatgt atatggagga aagaatgcgc cttacgtatg ggttcacttc |
| 1261 | ccgaaccaaa gctcatggga tgtgtttgct gagattctgg agaagactca tgtggttaca |
| 1321 | actccaggaa gtgggtttgg accagggggt gaagggttcg ttcgtgtcag tgcctttggt |
| 1381 | cacagagaga acatcttaga ggcatgtcga agattcaagc agctttacaa atgaagaacc |
| 1441 | ttgfftgtaa tcgttcctca tcatcatcac cctctttaat gacatgattt gagttaaaat |
| 1501 | aatgtcgttt ccattgtttt ctggaatttg tagaagacac ttttgacacc agtgtttcaa |
| 1561 | gcaatttggc aaagtatttt aacaacg |

### Results

### Identification of an LL-DAP-AT Activity in Plants

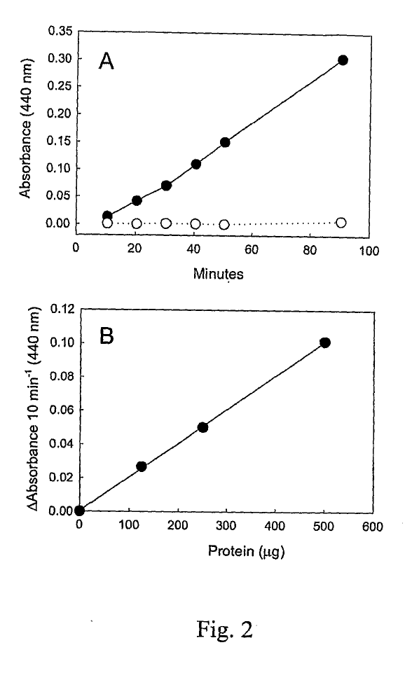

To search for an LL-DAP-AT activity in plants, an assay was developed to measure the production of THDPA using OAB, a compound that yields a dihyrodoquinazolinium adduct that has an absorbance maximum at A₄₄₀. When a soluble extract from axenically grown Arabidopsis plants was incubated with LL-DAP and 2-oxoglutarate (2-OG) as an amino acceptor, a linear formation of 440-nm absorbing material was observed over a period of 90 min (FIG. 2A). The rate of the reaction was directly proportional to the amount of protein extract added (FIG. 2B). No activity was observed if extract was omitted or when either LL-DAP or 2-OG were absent from the reaction. If the extract was heated in a boiling water bath for 5 min, the activity was completely destroyed. All of these observations strongly suggested that the activity was enzymatic. Moreover, since the source material was axenically grown, the activity must have been derived from the Arabidopsis rather than a contaminating microorganism.

Further analysis revealed that the enzyme activity is able to discriminate between isomers of DAP. See Table I. It was active only with LL-DAP and not its isomer m-DAP or two structurally related compounds Lys and Orn. The specificity for LL-DAP was further evidenced by the observation that m-DAP or Lys did not inhibit the use of LL-DAP, even when added at 1,000-fold excess concentration over LL-DAP (data not shown). The LL-DAP-AT was also able to discriminate between closely related keto acids (Table I). It used 2-OG as amino acceptor but was unable to use oxaloacetate or pyruvate. These results indicated that the LL-DAP-AT identified in Arabidopsis is highly specialized and a prime candidate for the enzyme that is involved in Lys synthesis.

TABLE I
Substrate specificity of Arabidopsis LL-DAP-AT

| Amino Donor/Acceptor | Activity |
|---|---|
| LL-DAP/2-OG | 20.1 |
| m-DAP/2-OG | <0.1 |
| Lys/2-OG | <0.1 |
| Orn/2-OG | <0.1 |
| LL-DAP/oxaloacetate | <0.1 |
| LL-DAP/pyruvate | <0.1 |

The assays were carried out as described in the legend to FIG. 2, except that amino donor and acceptor compounds were varied and 500 μg protein was assayed. Activity is ΔA (440 nm) min − 1 mg − 1 protein × 103. The minimum activity that could be confidently detected using the OAB assay was 0.1.

The taxonomic distribution of LL-DAP-AT activity was assessed to further evaluate whether it is possible that such an enzyme is generally involved in Lys biosynthesis in plants and their photosynthetic allies. Extracts prepared from a variety of vascular plants, from a moss, a green alga, and a cyanobacteria all showed LL-DAP-AT activity, whereas five bacterial species recognized as having one of the two known variants of the DAP pathways using acyl intermediates did not show LL-DAP-AT activity (Table II). The result of this limited taxonomic survey indicated that LL-DAP-AT activity is associated with photosynthetic organisms.

TABLE II
Taxonomic distribution of LL-DAP-AT activity

| Species | Activity |
|---|---|
| Arabidopsis ** | 20.1 |
| Brassica napus * | 2.1 |
| Chlamydomonas reinhardtii * | 14.5 |
| Convallaria majalis ** | 1.8 |
| Glycine max * | 5.2 |
| P. patens ** | 2.8 |
| Pea ** | 2.4 |
| Spinach * | 4.4 |
| Maize * | 9.3 |
| Synechococcus sp. * | 12.4 |
| Agrobacterium tumefaciens ** | <0.1 |
| Bacillus megaterium * | <0.1 |
| B. subtilis 168 ** | <0.1 |
| E. coli * | <0.1 |
| Rhizobium tropici ** | <0.1 |

The assay conditions were as described in FIG. 2, except that some samples were measured at 22° C. * and others at 30° C. **. Soluble protein extracts were prepared from the leaves of all the angiosperms except maize, which was prepared from embryogenic callus cultures. The other samples were from gametophytic thallus of P. patens or from growing cultures of the microorganisms. Activity is ΔA (440 nm) min⁻¹ mg protein⁻¹ × 10³.

Isolated chloroplasts are known to be capable of Lys synthesis from Asp (Mills and Wilson, 1978), indicating that all the enzymes of the pathway must reside within plastids. To determine whether LL-DAP-AT is contained within plastids, chloroplasts were purified from pea (Pisum sativum) by Percoll density gradient centrifugation. The activity from a soluble stromal extract was compared with the activity in a leaf extract. Pea was used for the experiment, rather than Arabidopsis, because chloroplast isolation is much more facile in this species. LL-DAP-AT activity was enriched 2.5-fold in the stromal extract compared with a leaf extract (data not shown), suggesting that the enzyme is contained, at least partly, within the soluble fraction of plastids. The experiment was not intended to determine whether the LL-DAP-AT activity exists in an extrachloroplast compartment.

### Identification of the Arabidopsis Gene Encoding LL-DAP-AT

Since the characterization of LL-DAP-AT would be greatly facilitated if the gene encoding this enzyme could be identified, a search was conducted of the Arabidopsis genomic loci encoding known and hypothetical aminotransferases. Specific sequence motifs have been defined that would allow aminotransferase genes to be readily identified in the DNA sequence databases. Using these characters, 44 likely aminotransferases were annotated in Arabidopsis (Liepman and Olsen, 2004). Of these, 19 were reported to be uncharacterized, in the sense that the specific reaction catalyzed by the gene product had not yet been established. Since LL-DAP-AT is very likely to be plastid localized, an initial focus was placed on five uncharacterized aminotransferases predicted to be localized to chloroplasts (At1g77670, At2g13810, At2g22250, At4g33680, and At5g57850). The corresponding cDNAs were expressed in Escherichia coli and an extract of each culture was assayed. In this way At4g33680 was identified as a genetic source of LL-DAP-AT activity. None of the other genes produced such an activity. At4g33680 (SEQ ID NO: 8) was annotated as a 461-amino acid (SEQ ID NO: 7), class I/II family aminotransferase. The first 36 amino acids were predicted by TargetP to be a transit peptide for localization of the protein to plastids. The closest paralog to At4g33680 in Arabidopsis is At2g13810, with which it shares 64.4% amino acid identity (Liepman and Olsen, 2004). Despite the homology, recombinant At2g13810 protein did not show LL-DAP-AT activity. It is important to emphasize that there are a number of explanations for why At2g13810 may not have shown LL-DAP-AT activity, but this question has not been explored yet.

### Quantitative Enzyme Assays and Properties of the Pure LL-DAP-AT

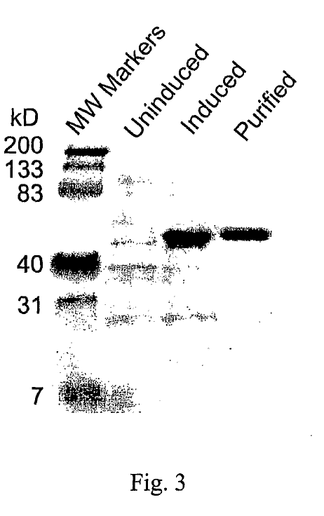

The kinetic properties of the pure recombinant At4g33680 enzyme were studied using several different assays. The expression and purification of LL-DAP-AT is shown in FIG. 3. The SDS-PAGE analysis shows that the At4g33680 expression plasmid produces a 51-kD protein, identical to the predicted molecular mass of the recombinant protein, and it is purified by nickel-affinity chromatography. The pure enzyme was found to have a 420-nm absorbance feature (data not shown) typically found in enzymes that have pyridoxal phosphate linked to a conserved Lys residue. Most aminotransferases require pyridoxal phosphate as a cofactor (Liepman and Olsen, 2004). The Lys residue at position 305 in the At4g33680 protein is predicted to be the pyridoxal phosphate ligand. With the reverse assay method using OAB the pure LL-DAP-AT showed the same substrate discrimination as the native enzyme in that it was specifically able to use LL-DAP as the amino donor and 2-OG as the acceptor (data not shown). The enzyme was also found to show a temperature optimum of 36° C. and a pH optimum of 7.6 when HEPESKOH buffer was used, and 7.9 when Tris HCl buffer was used (data not shown).

To examine the activity of LL-DAP-AT, the OAB assay was not ideal because the extinction coefficient of the dihyrodoquinazolinium adduct that OAB forms with THDPA was unknown and the assay would not be useful to determine whether LL-DAP-AT is able to function in the physiologically relevant direction. For this reason quantitative coupled assays were developed to assess the enzyme activity in both the reverse and forward directions. In the reverse direction the formation of THDPA by LL-DAP-AT was measured by coupling with Ddh from Corynebacterium glutamicum, which oxidizes NADPH when converting THDPA to m-DAP. The reaction sequence is shown in Scheme 1.

LL-DAP+2-OG→THDPA+Glu+water THDPA+NH₄⁺+NADPH→m-DAP+NADP⁺ (1)

Using this coupled-assay system, LL-DAP-AT was found to have an activity of 22.3 μmol min⁻¹mg⁻¹ protein and apparent Kₘ values of 67 μM for LL-DAP and 8.7 mM for 2-OG (Table III).

TABLE III
Kinetic properties of Arabidopsis LL-DAP-AT

| Assay | Vₘₐₓ | K_{cat} | Substrate | Kₘ | |
|---|---|---|---|---|---|
| Reverse | 22.3 ± 0.3 | 17.6 | LL-DAP | 67 ± 2 | μM |
| | | | 2-OG | 8.7 ± 0.3 | mM |
| Forward | 0.38 ± 0.01 | 0.3 | THDPA | 38 ± 4 | μM |
| | | | Glu | 1.9 ± 0.1 | mM |

The reverse reaction contained in 1 mL 100 μmol HEPESKOH (pH 7.5), 0.3 μmol NADPH, 50 μmol NH₄Cl, 0.5 μmol LL-DAP, 5 μmol 2-OG, 16 μg CtDdh, and pure LL-DAP-AT. The reaction was incubated at 30° C. and the decrease in A₃₄₀ was measured. The forward assay conditions were as described in the FIG. 2 legend. Vₘₐₓ is in μmol min⁻¹ mg⁻¹ protein and K_{cat} is in s⁻¹. The kinetic constants were calculated by nonlinear regression analysis using GraphPad Prizm version 4.03.

To measure the forward reaction, a coupled assay was developed that uses 2-OG dehydrogenase to assay 2-OG produced by aminotransfer from Glu to THDPA. To carry out this reaction, it was necessary to use NADPH-dependent Ddh to produce THDPA in situ. The overall reaction series is shown in Scheme 2.

m-DAP+NADP⁺→THDPA+NH₄⁺+NADPH THDPA+Glu+water→LL-DAP+2-OG 2-OG+CoA+thio-NAD⁺→succinylCoA+thio-NADH+CO₂+H⁺ (2)

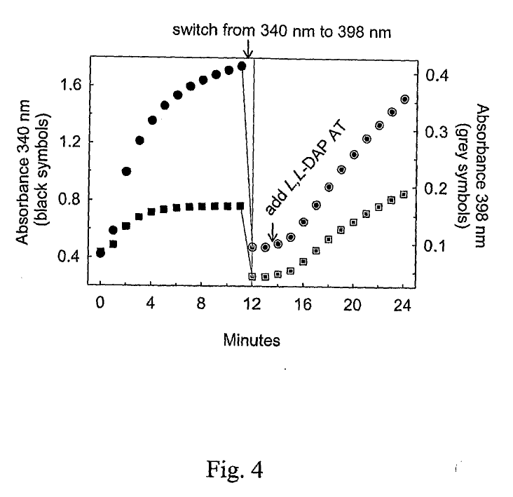

Due to the interference that NADPH formation would have on measurement of NADH produced by the 2-OG dehydrogenase reaction, it was necessary to replace NAD⁺ with thio-NAD⁺. Thio-NADH has an absorbance maximum at 398 nm, which can be discerned from NADPH, which has an absorbance maximum at 340 nm. FIG. 4 illustrates such a reaction. The black symbols show the progress of the THDPA-generating reaction monitored at 340 nm. Two plots are shown using 0.55 and 0.055 mM m-DAP. At the completion of the prereaction, the wavelength was changed to 398 nm, 2-OG dehydrogenase was added, and the absorbance monitored for 2 min. The decrease in absorbance when the wavelength was changed to 398 nm indicated that NADPH does not absorb light significantly at this wavelength. The unchanging absorbance after 2-OG dehydrogenase addition indicated that thio-NAD⁺ was not reduced by any combination of the substrates or the enzymes in the mixture. However, after addition of LL-DAP-AT there was a progressive increase in absorbance (gray symbols). The rate of the increase was dependent on the initial concentration of THDPA, which was formed from m-DAP in the prereaction. The results show that LL-DAP-AT can operate in the forward direction. Using this assay system, the specific activity of the LL-DAP-AT was found to be 0.38 μmol min⁻¹mg⁻¹ protein, and it showed an apparent Kₘ of 38 μM for THDPA and 1.9 mM for Glu (Table III). In total, the kinetic constants confirmed that LL-DAP-AT can catalyze the interconversion of THDPA and LL-DAP in vitro.

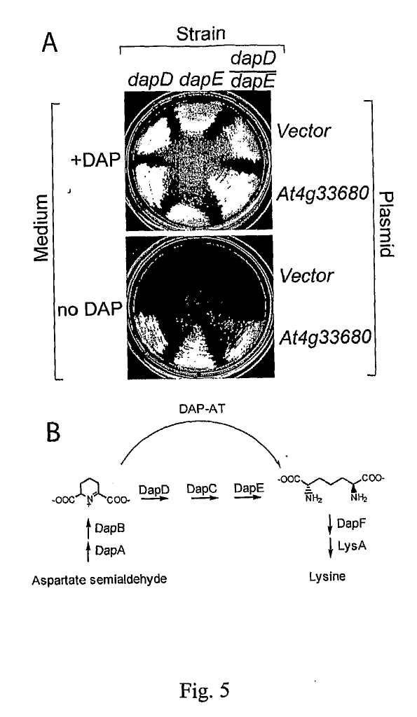

Given the unfavorable Vₘₐₓ in the forward reaction compared with the reverse reaction, it was of interest to examine whether this enzyme could drive Lys synthesis under physiological conditions. If LL-DAP-AT is able to directly convert THDPA into LL-DAP, it has the potential to bypass the three separate enzymes needed to catalyze the same overall reaction in E. coli, the products of the dapD, dapC, and dapE genes. Of these, only dapD and dapE mutants are auxotrophic for DAP and suitable for the functional complementation assay. The dapD and dapE mutant strains and a double dapD/dapE mutant were transformed with either an empty plasmid or an At4g33680 expression plasmid. FIG. 5A shows that, while all strains were able to grow on medium containing DAP, only the strains carrying the At4g33680-expressing plasmid were able to grow without DAP, indicating that the enzyme encoded by At4g33680 is able to bypass the succinylation and desuccinylation reactions required by E. coli to synthesize LL-DAP from THDPA (FIG. 5A). By contrast, At4g33680 was unable to complement adapB mutant (data not shown). The complementation result confirms that LL-DAP-AT can function in the forward direction under physiological conditions by catalyzing in a single step, a reaction that requires three enzymes in E. coli (FIG. 5B).

### Phylogenetic Distribution of LL-DAP-AT and the Plant-Type Lys Biosynthesis Pathway

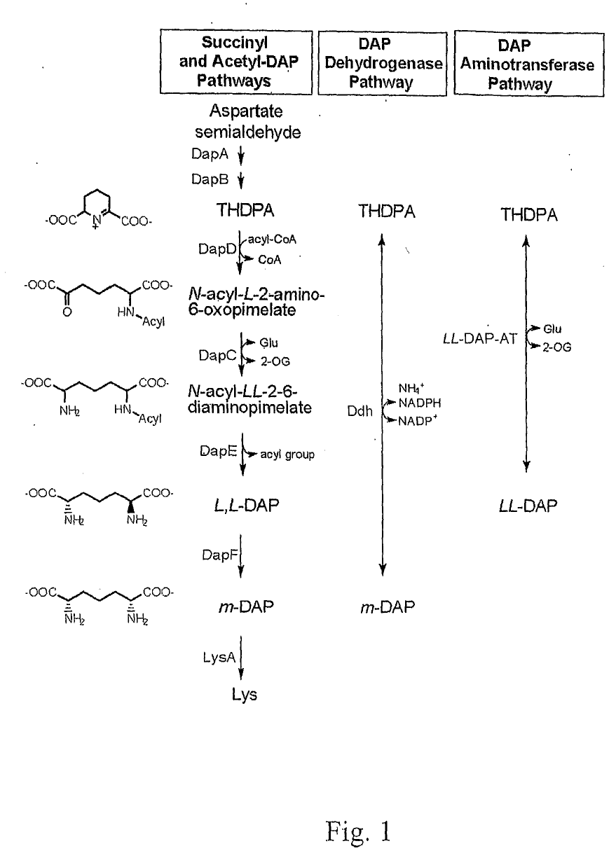

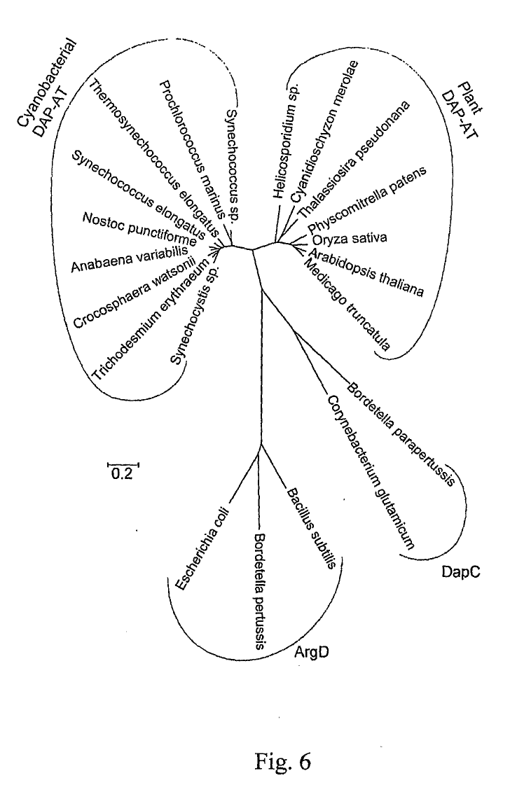

To assess the taxonomic distribution of LL-DAP-AT, the coding sequence of At4g33680 was used to search the protein sequence databases. A neighbor-joining tree showing the relationship of homologous sequences in plant and cyanobacteria is depicted in FIG. 6. For the sake of comparison the DapC sequences from Bordetella parapertussis, C. glutamicum, and the ArgD sequences from E. coli, Bordetella pertussis, and Bacillus subtilis were included in the analysis. Both DapC and ArgD have been shown to catalyze aminotransfer to N-succinyl-L-2-amino-6-oxopimelate, which is the reaction in the acyl DAP pathways (Ledwidge and Blanchard, 1999; Fuchs et al., 2000; Hartmann et al., 2003) analogous to that catalyzed by LL-DAP-AT (FIG. 1). The tree shows three major clades. One includes the orthologs of LL-DAP-AT, which branches into closely related cyanobacterial and eukaryotic forms. Another includes DapC orthologs, and a third includes ArgD orthologs. The clades share low sequence homology. For example, Synechocystis sll0480 shares about 19% sequence identity with either C. glutamicum or B. parapertussis DapC. In contrast, the most divergent members of the LL-DAP-AT group share a minimum of 46% identity. By way of comparison, the closest sll0480 homologs in C. glutamicum (encoded by locus NCgl0780) and B. parapertussis (encoded by locus BPP2478) show about 23% identity with sll0480. Thus, despite the fact that class I and II family aminotransferases are descended from a common ancestor, the divergence indicates that the different LL-DAP-AT of the invention share a distinct lineage that is different from either DapC or ArgD. To assess the function of the cyanobacterial subgroup of the LL-DAP-AT clade, the ortholog from Synechocystis (sll0480) was cloned and expressed in E. coli. Like its Arabidopsis counterpart, sll0480 exhibited robust LL-DAP-AT activity approximately equal to recombinant At4g33680 and was able to functionally complement E. coli dapD, dapE, and dapD/dapE mutants (data not shown). The finding that sll0480 encodes an LL-DAP-AT suggests that Synechocystis and very likely all the cyanobacteria may have a Lys biosynthesis pathway similar to that found in plants. To obtain additional evidence for this hypothesis, the cyanobacterial sequenced genomes were surveyed for orthologs of the known DAP proteins from heterotrophic bacteria. If cyanobacteria have a plant-like Lys biosynthesis pathway, they would be expected to lack orthologs of DapD, DapC, DapE, and Ddh, just as was recently observed for Arabidopsis (Hudson et al., 2005). No orthlogs of DapD, DapE, and Ddh could be identified in any of the sequenced cyanobacterial genomes (See Table IV below cataloging the Dap genes in Synechocystis sp.). Although DapC orthlogs could be identified in all the cyanobacterial species, the level of homology (approximately 20% identity) was well below the 45% identity observed among the most divergent LL-DAP-AT orthologs. These results indicate that cyanobacteria, like Arabidopsis, probably lack the enzymes from the core of the acyl-DAP and Ddh pathways. This observation, coupled with the functional identification of an LL-DAP-AT from Synechocystis, suggests that cyanobacteria synthesize Lys via an enzyme that directly converts THDPA to LL-DAP.

TABLE IV
Synechocystis sp. was searched with blastp using the annotated DAP enzymes from
Corynebacterium glutamicum or Bordetella parapertussis as query. These species were
chosen as query because the DAP pathway has been well characterized in them and
C. glutamicum was also chosen because it contains both the acyl-DAP and Ddh pathways for
lysine synthesis. Corynebacterium contains two paralogous proteins for DapD and LysA and
each paralog was used separately as query. The query (grey shaded boxes) and retrieved
sequences (unshaded boxes) are indicated by their associated locus codes, NCg =
Corynebacterium, BPP = Bordetella, slr or sll = Synechocystis. The blastp homology values
are shown below the retrieved protein (unshaded boxes). For the sake of comparison the DapC
queries were also compared by blastp with sll0480, the protein identified as the DAP AT in
Synechacystis.

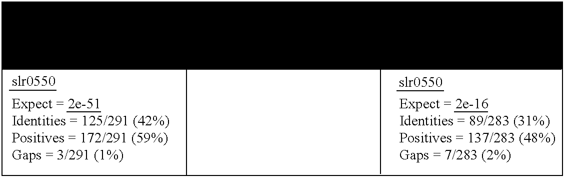

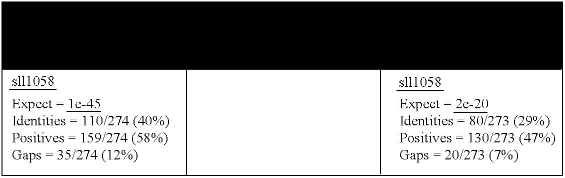

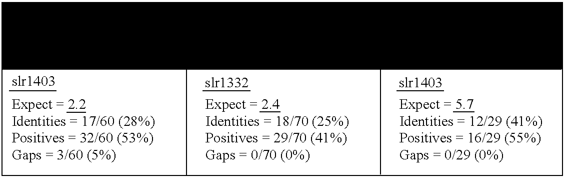

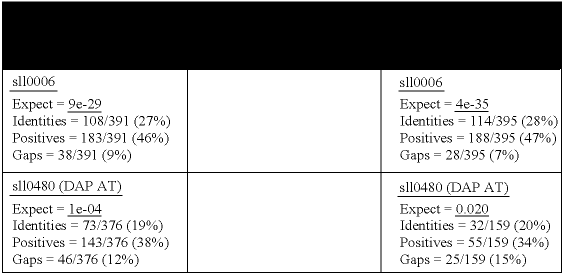

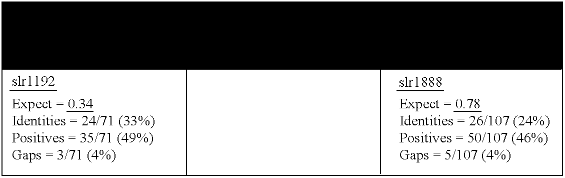

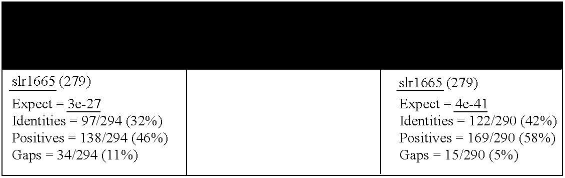

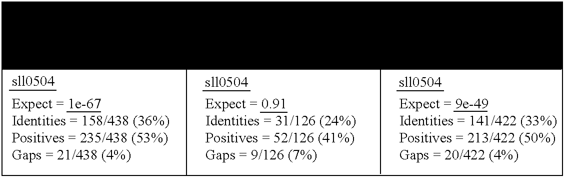

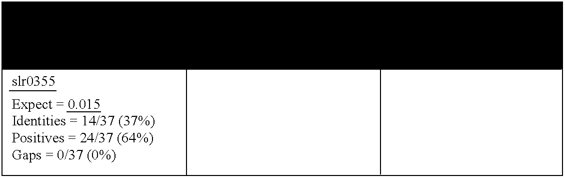

The results in the supplementary table show that DapA, DapB, DapF and LysA orthologs can be readily identified in Synechocystis sp. based on homology with Corynebacterium and Bordetella coding sequences for these proteins. DapD and DapE orthologs cannot be identified. The best matches have E scores that are no greater than randomly selected sequences. DapC orthologs can be identified. The best match to Corynebacterium DapC shows an E value of 9e-29 and to Bordetella DapC, 4e-35. The Corynebacterium and Bordetella DapC show much lower homology to the LL-DAP aminotransferase sll0480, and the DapC orthologs in Synechocystis show lower homology than the most divergent LL-DAP aminotransferase orthologs (e.g. sll0480 and At4g33680 show an E value of 7.0e-86). In total, the results suggest that Synechocystis lacks the acyl and Ddh pathways for lysine synthesis and have an LL-DAP aminotransferase pathway.

Assays for assessing the ability of certain compounds to inhibit LL-DAP aminotransferase have been performed and the results are shown below in Table V. The reactions were performed as described above.

TABLE V
Inhibitors of LL-DAP Transaminase

| Inhibitor | Conc. mM | Substrate | Conc. mM | Inhibition % |
|---|---|---|---|---|
| L, D-α- | 5 | LL-DAP | 0.5 | 50+ |
| aminopimelate | | | | |
| | 10 | | 0.5 | 83+ |
| Threo-β-hydroxy | 40 | LL-DAP | 0.05 | 68++ |
| DAP (racemic) | | | | |

+Ortho aminobenzaldehyde Assay (maize transaminase)
++m-DAP dehydrogenase Coupled Assay (A. thaliana transaminase)

### Discussion

The existence of a novel variant of the DAP pathway was predicted in Arabidopsis based on the finding that this species does not contain an apparent ortholog of DapD nor functional homologs of DapC and DapE (Hudson et al., 2005). Since these enzymes form the core of the prokaryotic acyl pathway for Lys synthesis, their absence and the previous demonstration that a number of plants do not contain Ddh (Chatterjee et al., 1994) raised the question that prompted this study: How do plants bridge the metabolic gap between THDPA and LL-DAP? The findings reported here answer this question. They do so with a single enzyme, an aminotransferase that directly converts THDPA to LL-DAP. This finding adds yet another example to the list of natural variants of the DAP pathway.

LL-DAP-AT is able to bypass the acylation and deacylation steps found in most bacteria. To our knowledge, the function of acylation in the biosynthesis of DAP has never been clearly delineated. The equilibrium between the cyclic and acyclic structures favors THDPA, yet it is the acyclic form that contains the keto group needed for transamination. For this reason, it was proposed that acylation speeds the conversion of the ring-structured THDPA to the acyclic form (Berges et al., 1986). The DapD enzyme was envisioned as adding water to the imine of THDPA to produce a trans-piperidine dicarboxylate intermediate to which the acyl group is added, thereby facilitating ring opening. Therefore, in species with an LL-DAP-AT pathway, the rate of Lys synthesis would depend on the spontaneous rate of ring opening unless the process was catalyzed. Whether LL-DAP-AT catalyzes THDPA ring opening remains to be investigated.

Much evidence exists supporting the idea that chloroplasts were derived from an endosymbiosis between a cyanobacterium and aheterotrophic, mitochondrion-containing eukaryote (Falkowski et al., 2004). After the symbiosis the cyanobacterial genes were subsequently transferred to the host nucleus, where they acquired the sequences necessary to target the proteins to the chloroplast (Martin et al., 2002). The conservation and taxonomic distribution of the LL-DAP-AT in eukaryotic photoautotrophs and cyanobacteria are consistent with a cyanobacterial origin of plastids.

At4g33680, the locus encoding LL-DAP-AT, was previously identified based on the phenotype of a point mutant that caused aberrant growth defects and cell death named agd2 (Song et al., 2004). Based on their finding that the AGD2 protein was able to transaminate Lys with a physiologically implausible Kₘ of 58.8 mM, Song et al. (2004) proposed that it might be involved in Lys metabolism. In fact, as reported here the Kₘ for LL-DAP is 830-fold lower than the value for Lys. In addition, a 1,000-fold excess addition of Lys to an assay did not inhibit LL-DAP-AT activity (data not shown). Thus, Lys itself is not a substrate for, nor does it inhibit LL-DAP-AT. In seeking a plausible explanation for these disparate observations, it did seem possible that with the very high concentrations of Lys (100 mM) used by Song et al. (2004) that even small amounts of contaminants might have given rise to their results. Since commercially available Lys is prepared by bacterial fermentation, it was not surprising to find that analysis of our own Lys stock revealed the presence of detectable amounts of LL-DAP and m-DAP.

The data reported here indicate that At4g33680 encodes an LL-DAP-AT that can function in Lys synthesis. Whether it is the only enzyme that can convert THDPA to LL-DAP in Arabidopsis is not absolutely known. Although its closest paralog in Arabidopsis, encoded by At2g13810, did not show LL-DAP-AT activity when expressed in E. coli, it is important to mention that this negative evidence does not rule out the possibility that it has this activity. However, a T-DNA-insertional, knockout allele of At4g33680 has been found to be embryo lethal, indicating that this gene is essential (Song et al., 2004). Further analysis will be necessary to resolve the question of whether At4g33680 is a unique gene or is a member of a functionally redundant gene family.

The initial identification of LL-DAP-AT was made by measuring the conversion of LL-DAP to THDPA, a reaction that runs in the reverse direction relative to Lys synthesis. The activity of the enzyme proved to be highly specific in that it was able to distinguish between DAP isomers and several acceptors commonly used by aminotransferases. The LL-DAP-AT was unable to use M-DAP, an isomer of LL-DAP. In addition, 2-OG was used as amino acceptor specifically over pyruvate and oxaloacetate. LL-DAP-AT also proved to be capable of the physiologically significant forward activity with an initial rate that is disfavored by 50-fold compared with the reverse activity. Despite this unfavorable feature, the enzyme was demonstrated to function in the forward direction under physiological conditions by the fact that it is able to substitute for the lack of succinyltransferase and deacylase activities in the dapD and dapE mutants of E. coli. Barring the possibility that the molecular construction used to produce the recombinant enzyme negatively affected its catalytic properties, it is very likely that the physiological concentrations of substrates offset the unfavorable Vₘₐₓ ratio. The level of Glu in the chloroplast stroma has been reported for several plant species to be in the range of 14 to 73.6 mM (Winter et al., 1993, 1994; Leidreiter et al., 1995), well above the 2.0 mM K_{m[Glu]} of the LL-DAP-AT. Although 2-OG concentration has been less well documented, it was found to be 70 μM in the chloroplast stroma of spinach (Spinacia oleracea) and has been estimated, based on the properties of a 2-OG/malate transporter in Arabidopsis, to be in the low micromolar range (Weber and Flugge, 2002). Such a concentration is well below the 8.3 mM K_{m[2-OG]} of the LL-DAP-AT. The E. coli cytoplasm also contains a high Glu/2-OG ratio (Cayley et al., 1991), which, just as in the chloroplast stroma, drives biosynthetic transaminations, and explains why LL-DAP-AT can replace DapD and DapE in E. coli. A further indication that the conditions in plastids favor the forward reaction for LL-DAP-AT comes from measurement of the activities of enzymes acting before and after the LL-DAP-AT. In extracts prepared from maize (Zea mays) embryo cultures, the LL-DAP-AT forward activity (estimated based on the ratio of forward and reverse activity of the pure Arabidopsis recombinant enzyme) was about 0.16 nmol min⁻¹mg⁻¹ protein. Extracts from the same culture showed DapA activity of 1.3 nmol min⁻¹mg⁻¹ protein, DapB activity of 17.0, DapF activity of 29.0, and LysA activity of 48.0 (Chatterjee et al., 1994). All of these activities are an order of magnitude or more above the activity of the LL-DAP-AT. Although the concentrations of THDPA and LL-DAP have never been directly measured in plants, based on the enzyme-specific activities it is likely that the THDPA concentration would be higher than that of LL-DAP. Another interesting observation that can be gleaned from the analysis of Lys biosynthesis enzyme activities from maize embryo cultures is that the overall rate of Lys synthesis was 0.53 nmol min⁻¹mg⁻¹ protein (Hudson et al., 2005), in the same range as the forward rate of LL-DAP-AT (calculated from the OAB activity in Table 1, based on the ratio of forward and reverse activity of the pure enzyme in Table III). Thus, it appears that in the case of the maize embryo culture, LL-DAP-AT may limit the biosynthesis of Lys under some conditions. It is recognized that the first enzyme of the pathway, dihydrodipicolinate synthase, is feedback regulated by Lys and plays a primary role in regulating the pathway (Shaul and Galili, 1993). LL-DAP-AT activity may play a role in limiting the pathway when dihydrodipicolinate synthase is not inhibited by Lys.

The discovery of LL-DAP-AT and the hint that it may be a factor limiting the rate of Lys biosynthesis could have implications for agriculture. Animals cannot produce Lys and so they rely on a dietary source, which is derived primarily from crop plants. Since some crops do not accumulate enough Lys to allow them to be used as complete nutritional sources, there has been significant interest in improving nutritional quality by enhancing Lys content (Mazur et al., 1999). It is known that in plants the control of Lys homeostasis is complex with degradation playing as significant a role as biosynthesis (Galili et al., 2001, Zhu and Galili, 2004). Therefore, the discovery of LL-DAP-AT has completed our understanding of the exact pathway by which plants synthesize Lys and has revealed another potential target for plant improvement.

### REFERENCES FROM EXAMPLE I

Azevedo R A (2003) Analysis of the aspartic acid metabolic pathway using mutant genes. Amino Acids 22: 217-230
Berges D A, DeWolf W E Jr, Dunn G L, Newman D J, Schmidt S J, Taggart J J, Gilvarg C (1986) Studies on the active site of succinyl-CoA:tetrahydrodipicolinate N-succinyltransferase: characterization using analogs of tetrahydrodipicolinate. J Biol Chem 261: 6160-6167
Caplan J F, Sutherland A, Vederas J C (2001) The first stereospecific synthesis of L-tetrahydrodipicolinic acid; a key intermediate of diaminopimelate metabolism. J Chem Soc Perkin Trans 1: 2217-2220
Cayley S, Lewis B A, Guttman H J, Record M T Jr (1991) Characterization of the cytoplasm of Escherichia coli K-12 as a function of external osmolarity: implications for protein-DNA interactions in vivo. J Mol Biol 222: 281-300
Chatterjee S P, Singh B K, Gilvarg C (1994) Biosynthesis of lysine in plants: the putative role of meso-diaminopimelate dehydrogenase. Plant Mol Biol 26: 285-290
Chrystal E J T, Couper L, Robins D J (1995) Synthesis of a key intermediate in the diaminopimelate pathway to L-Lysine: 2,3,4,5-tetrahydrodipicolinic acid. Tetrahedron 51:10241-10252
Cox R J, Wang P S H (2001) Is N-acetylornithine aminotransferase the real N-succinyl-LL-diaminopimelate aminotransferase in Escherichia coli and Mycobacterium smegmatis? J Chem Soc Perkin Trans 1: 2006-2008
Cranenburgh R M, Hanak J A, Williams S G, Sherratt D J (2001) Escherichia coli strains that allow antibiotic-free plasmid selection and maintenance by repressor titration. Nucleic Acids Res 29: E26
Falkowski P G, Katz M E, Knoll A H, Quigg A, Raven J A, Schofield O, Taylor F J (2004) The evolution of modern eukaryotic phytoplankton. Science 305: 354-360
Fuchs T M, Schneider B, Krumbach K, Eggeling L, Gross R (2000) Characterization of a Bordetella pertussis diaminopimelate (DAP) biosynthesis locus identifies dapC, a novel gene coding for an N-succinyl-L,L-DAP aminotransferase. J Bacteriol 182: 3626-3631
Galili G, Tang G, Zhu X, Gakière B (2001) Lysine catabolism: a stress and development super-regulated metabolic pathway. Curr Opin Plant Biol 4: 261-266
Gilvarg C (1959) N-Succinyl-L-diaminopimelic acid. J Biol Chem 234: 2955-2959
Gilvarg C (1961) N-Succinyl-alpha-amino-6-ketopimelic acid. J Biol Chem 236: 1429-1431
Gorman D S, Levine R P (1966) Cytochrome f and plastocyanin: their sequence in the photoelectric transport chain. Proc Natl Acad Sci USA 54: 1665-1669
Guzman L M, Belin D, Carson M J, Beckwith J (1995) Tight regulation, modulation, and high-level expression by vectors containing the arabinose PBAD promoter. J Bacteriol 177: 4121-4130
Hartmann M, Tauch A, Eggeling L, Bathe B, Mockel B, Puhler A, Kalinowski J (2003) Identification and characterization of the last two unknown genes, dapC and dapF, in the succinylase branch of the L-lysine biosynthesis of Corynebacterium glutamicum. J Biotechnol 104: 199-211
Hudson A O, Bless C, Macedo P, Chatterjee S P, Singh B K, Gilvarg C, Leustek T (2005) Biosynthesis of lysine in plants: evidence for a variant of the known bacterial pathways. Biochim Biophys Acta 1721: 27-36
Koncz C, Schell J (1986) The promoter of TL-DNA gene 5 controls the tissue specific expression of chimeric genes carried by a novel type of Agrobacterium binary vector. Mol Gen Genet. 204: 383-396
Kumar S, Tamura K, Jakobsen I B, Nei M (2001) MEGA2: molecular evolutionary genetics analysis software. Bioinformatics 17: 1244-1245
Ledwidge R, Blanchard J S (1999) The dual biosynthetic capability of N-acetylomithine aminotransferase in arginine and lysine biosynthesis. Biochemistry 38: 3019-3024
Leidreiter K, Kruse A, Robinson D, Heldt H (1995) Subcellular volumes and metabolite concentrations in potato (Solanum tuberosum cv. Desiree) leaves. Bot Acta 108: 439-444
Liepman A H, Olsen L J (2004) Genomic analysis of aminotransferases in Arabidopsis thaliana. CRC Crit. Rev Plant Sci 23: 73-89
Martin W, Rujan T, Richly E, Hansen A, Comelsen S, Lins T, Leister D, Stoebe B, Hasegawa M, Penny D (2002) Evolutionary analysis of Arabidopsis, cyanobacterial, and chloroplast genomes reveals plastid phylogeny and thousands of cyanobacterial genes in the nucleus. Proc Natl Acad Sci USA 99: 12246-12251
Matthews B F (1999) Lysine, Threonine, and Methionine Biosynthesis. Marcel Dekker, New York
Mazur B, Krebbers E, Tingey S (1999) Gene discovery and product development for grain quality traits. Science 285: 372-375
Mills W R, Wilson K G (1978) Amino acid biosynthesis in isolated pea chloroplasts: metabolism of labeled aspartate and sulfate. FEBS Lett 92: 129-132
Misono H, Togawa H, Yamamoto T, Soda K (1976) Occurrence of meso-alpha, epsilon-diaminopimelate dehydrogenase in Bacillus sphaericus. Biochem Biophys Res Commun 72: 89-93
Richaud C, Higgins W, Mengin-Lecreulx D, Stragier P (1987) Molecular cloning, characterization, and chromosomal localization of dapF, the Escherichia coli gene for diaminopimelate epimerase. J Bacteriol 169: 1454-1459
Sambrook J, Fritsch E, Maniatis T (1989) Molecular Cloning: A Laboratory Manual, Ed 2. Cold Spring Harbor Laboratory Press, Cold Spring Harbor, N.Y.
Schaefer D, Zryd J P, Knight C D, Cove D J (1991) Stable transformation of the moss Physcomitrella patens. Mol Gen Genet. 226: 418-424
Schöpf C, Steuer H (1947) Zur frage der biogenese der rutaecarpins und evodiamins. Liebigs Ann Chem 558: 124-136
Shaul O, Galili G (1993) Concerted regulation of lysine and threonine synthesis in tobacco plants expressing bacterial feedback-insensitive aspartate kinase and dihydrodipicolinate synthase. Plant Mol Biol 23: 759-768
Singh B K, Stidham M, Shaner D (1988) Separation and characterization of two forms of acetohydroxy acid synthase from Black Mexican Sweet corn cells. J Chromatog 444: 251-261
Song J T, Lu H, Greenberg J T (2004) Divergent roles in Arabidopsis thaliana development and defense of two homologous genes, aberrant growth and death2 and AGD2-LIKE DEFENSE RESPONSE PROTEIN1, encoding novel aminotransferases. Plant Cell 16: 353-366
Sundharadas G, Gilvarg C (1967) Biosynthesis of alpha,epsilon-diaminopimelic acid in Bacillus megaterium. J Biol Chem 242: 3983-3984
Velasco A M, Leguina J I, Lazcano A (2002) Molecular evolution of the lysine biosynthetic pathways. J Mol Evol 55: 445-459
Vogel H J (1959) On biochemical evolution: lysine formation in higher plants. Proc Natl Acad Sci USA 45: 1717-1721
Weber A, Flugge U I (2002) Interaction of cytosolic and plastidic nitrogen metabolism in plants. J Exp Bot 53: 865-874
Wehrmann A, Eggeling L, Sahm H (1994) Analysis of different DNA fragments of Corynebacterium glutamicum complementing dapE of Escherichia coli. Microbiology 140: 3349-3356
Weinberger S, Gilvarg C (1970) Bacterial distribution of the use of succinyl and acetyl blocking groups in diaminopimelic acid biosynthesis. J Bacteriol 101: 323-324
White P J (1983) The essential role of diaminopimelate dehydrogenase in the biosynthesis of lysine by Bacillus sphaericus. J Gen Microbiol 129: 739-749
Winter H, Robinson D G, Heldt H W (1993) Subcellular volumes and metabolite concentrations in barley leaves. Planta 191: 180-190
Winter H, Robinson D G, Heldt H W (1994) Subcellular volumes and metabolite concentrations in spinach leaves. Planta 193: 530-535
Zhu X, Galili G (2004) Lysine metabolism is concurrently regulated by synthesis and catabolism in both reproductive and vegetative tissues. Plant Physiol 135: 129-136

### Example II

### A New Variant of the Lysine Biosynthesis Pathway that the Chlamydiales have in Common with Plants is Defined by an LL-Diaminopimelate Aminotransferase Encoded by ct390 in Chlamydia trachomatis

The synthesis of meso-diaminopimelic acid (m-DAP) is necessary for most bacteria for its use for lysine biosynthesis and for peptidoglycan (PG) (1). In contrast, animals neither synthesize nor utilize M-DAP as a substrate in any metabolic pathway and lysine is an essential amino acid that is obtained from dietary sources (2-4).

m-DAP/lysine synthesis comprises a branch of the aspartate metabolic pathway which also includes the synthesis of methionine, threonine and isoleucine (FIG. 1). Common to the synthesis of all these amino acids is the conversion of L-aspartate to L-aspartate-semialdehyde via LysC and Asd (5, 6). The first reaction unique to m-DAP/lysine synthesis is the DapA-catalyzed condensation of L-aspartate-semialdehyde and pyruvate to generate dihydrodipicolinate, which is subsequently reduced by DapB to tetrahydrodipicolinate (THDPA). Hereafter, we refer to the four-step synthesis of THDPA as the upper m-DAP synthesis pathway. From THDPA, three variant pathways have been defined for m-DAP synthesis in the bacteria (the succinylase, acetylase, and dehydrogenase pathways). The succinylase pathway uses succinylated intermediates and is the most widely distributed in bacteria. Genes encoding THDPA succinyltransferase (dapD) and N-succinyl-LL-DAP desuccinylase (dapE) have been characterized while two enzymes, DapC and ArgD, have been shown to possess N-succinyl-LL-DAP aminotransferase (AT) activity (7-11). An analogous pathway using acetylated intermediates has been biochemically detected in certain Bacillus spp. yet genes in this branch have not been characterized (12). The final step in both of the acyl pathways is the epimerization of LL-DAP to M-DAP by DapF. A third pathway exists in which a DAP dehydrogenase directly converts THDPA to m-DAP. The dehydrogenase pathway is utilized by a small number of gram-positive organisms and in some cases, in conjunction with an acylase pathway. Collectively, we term the steps that convert THDPA to m-DAP as the lower m-DAP biosynthesis pathway. Once m-DAP is synthesized, it is either incorporated into PG or decarboxylated to lysine by LysA.

Bacteria of the order Chlamydiales are obligate intracellular bacteria that are pathogenic for humans and animals and lack detectable PG. Despite the inability to detect PG, a nearly complete and functional PG pathway is encoded in the chlamydiae genomes (13). However, the m-DAP synthesis pathway in chlamydiae is incomplete (FIG. 1). The upper m-DAP pathway is intact as lysC, asd, dapA and dapB homologues are present in these genomes, yet no homologues of any of the lower m-DAP pathways have been annotated with the exception of dapF. Furthermore, no lysA gene has been detected in the chlamydial genomes despite the biochemical detection of lysine decarboxylase activity in C. psittaci (14).

The following materials and methods are provided to facilitate the practice of Example II.

Nucleic Acid Encoding LL-DAP-AT from Chlamydia

The availability of the sequence information for LL-DAP-AT from Arabidopsis thaliana facilitated performance of a search of the sequence data bases to identify homologs and orthologs of the enzyme from other species. This search revealed an open reading frame from Chlamydia, ct390 which appeared to encode a similar aminotransferase enzyme. See GenBank accession No. NC-000117 wherein the sequence of ct390 is disclosed. The amino acid sequence for the enzyme is set forth below as SEQ ID NO: 9. The nucleic acid encoding the enzyme was cloned using conventional procedures. See Current Protocols in Molecular Biology. Ausubel et al. eds. (JW Wiley & Sons).

| | |
|---|---|
| MKRNPHFVSLTKNYLFADLQKRVAQFRLENPQHTVIN | SEQ ID NO: 9 |
| LSIGDTTQPLNASVAEAFASSIARLSSPTTCRGYGPD | |
| FGLPALRQKLSEDFYRGFVDAKEIFISDGAKVDLFRL | |
| LSFFGPNQTVAIQDPSYPAYLDIARLTGAKEIIALPC | |
| LQENAFFPEFPEDTHIDILCLCSPNNPTGTVLNKDQL | |
| RAIVHYAIEHEILILFDAAYSTFISDPSLPKSIFEIP | |
| DARFCAIENSFSKPLGFAGIRLGWTVIPQELTYADGH | |
| FVIQDWERFLSTTFNGASIPAQEAGVAGLSILPQLEA | |
| IHYYRENSDLLRKALLATGFEVFGGEHAPYLWVKPTQ | |
| ANISDRDLFDFFLREYHIAITPGIGFGRSGSGFVRFS | |
| SLGKREDILAACERLQMAPALQS | |

Enzyme Assays

LL-DAP and m-DAP were synthesized and purified as described in Gilvarg (1959). Corynebacterium glutamicum diaminopimelate dehydrogenase (CgDdh) was produced as a recombinant protein expressed from plasmid pET28-CgDDH obtained from D. I. Roper (University of Warwick) in E. coli BL21(DE3). Ddh was expressed to approximately 90% of the soluble protein and converted M-DAP to THDPA at a rate of 14 μmol min⁻¹mg⁻¹ protein at 30° C. so it was not further purified for use in enzyme assays.

All enzyme assays were carried out essentially as described in Example I. Incubation temperature was 30° C. Kinetic constants were determined by varying substrate concentrations while keeping the co-substrate level constant at the concentration described below. Kinetic data were analyzed by non-linear regression analysis using GraphPad Prizm Version 4.03.

For LL-DAP aminotransferase measurement of pure enzyme a quantitative assay for the reverse activity was carried out in 1 mL containing 100 μmol HepesKOH (pH 7.5), 0.3 μmol NADPH, 50 μmol NH₄Cl, 0.5 μmol LL-DAP, 5 μmol 2-oxoglutarate (2-OG), 16 μg CgDdh, and pure CT390. The background rate was measured in a reaction lacking either LL-DAP or 2-OG.

Quantitative assay of the physiologically forward reaction was carried out sequentially in a pre-reaction to generate THDP from m-DAP followed by an LL-DAP aminotransferase assay. The pre-reaction contained in slightly less than 1 mL, 100 μmol HepesKOH (pH 7.5), 0.5 μmol NADP⁺, 0.5 mM m-DAP, 32 g CgDdh, 0.3 μmol thio-NAD⁺, 0.3 μmol CoA, and 0.5 μmol Glu. The pre-reaction was run to completion, monitored by the increase in absorbance at 340 nm resulting from NADPH formation. The aminotransferase reaction was monitored at 398 nm to measure the formation of thioNADH. The background rate was determined after adding 200 μg of 2-oxoglutarate dehydrogenase (0.625 μmol min⁻¹mg⁻¹ protein). Subsequently, the aminotransferase assay was initiated by addition of CT390, bringing the total reaction volume to 1 mL.

A semi-quantitative assay of the reverse activity was used for measuring LL-DAP aminotransferase activity in crude protein extracts. Soluble proteins were extracted from E. coli by sonication in 100 mM HepesKOH (pH 7.6) followed by buffer exchange using an Amicon Ultra 30,000 MWCO filter. The reaction contained in 1 mL, 100 tμmol HepesKOH (pH 7.6), 0.5 μmol amino donor, 2.0 μmol 2-OG, and 1.25 mg OAB, and crude soluble protein or pure protein. Reactions were monitored at 440 nm.

### Results

### Analysis of ct390 Function

To determine how the chlamydiae metabolize THDP the C. trachomatis genomic DNA was searched for sequences having high levels of similarity to At4g33680. This search revealed an open reading frame designated as ct390 which appeared to encode an LL-DAP-AT. The nucleic acid encoding the ORF was expressed and the enzyme purified.

### Enzyme Kinetics

To further examine the function of CT390 the protein was expressed in E. coli as a fusion with a His-Tag for purification by Ni affinity chromatography. Spectral analysis of pure CT390 revealed the presence of an absorbance feature centered at about 420 nm, indicative of the presence of pyridoxal phosphate (PLP) (data not shown). All transaminases use PLP as a co-factor and the CT390 amino acid sequence shows a canonical PLP binding site including the PLP ligand at Lys 236. Most aminotransferases catalyze reversible reactions. To determine whether this is true for CT390, its activity was studied using coupled assay systems for measurement of the catabolic reverse reaction and the biosynthetically relevant forward reaction. In the forward reaction the enzyme efficiently transferred an amino group from glutamate to THDP. At saturating concentrations of the substrates the Vₘₐₓ was 0.38 μmol min⁻¹mg proteins⁻¹ and the apparent Kₘ values were 200 μM for THDP and 1.0 mM for glutamate (Table VI). In the reverse direction, CT390 efficiently transferred an amino group from LL-DAP to 2-oxoglutarate. At saturating concentrations of the substrates the Vₘₐₓ was 0.59 μmol min⁻¹mg proteins⁻¹ and the apparent Kₘ values were 20 μM for LL-DAP and 0.65 mM for 2-OG (Table VI). Interestingly, CT390 was able to use m-DAP as an amino donor in the reverse direction. However it was unable to use ornithine, lysine, or cystathionine as amino donors. These results indicate that CT390 is a transaminase that can specifically synthesize LL-DAP from THDP. The ability to use m-DAP for the reverse reaction distinguishes CT390 from the recently characterized plant LL-DAP aminotransferase, which was specific for LL-DAP (15). Although the basis for the relaxed substrate specificity is presently not known it is unlikely to be of physiological significance since D-THDP would be produced from m-DAP rather than L-THDP. D-THDP would be a metabolic dead-end since it could not be used in the forward reaction.

TABLE VII
Kinetic properties of CT390

| Assay | Vₘₐₓ | K_{cat} | Substrate | Kₘ | |
|---|---|---|---|---|---|
| | | | L,L-DAP | 20 | μM |
| Reverse | 0.59 | | 2-OG | 0.65 | mM |
| | | | THDPA | 200 | μM |
| Forward | 0.38 | | Glu | 1.0 | mM |

Homologs of CT390 are highly conserved. Orthologous sequences are found in all of the chlamydial species whose genomes have been sequenced. The most divergent representative is Protochlamydia amoebophila orf pc0685 with which it shares 42.6% amino acid sequence identity. P. amoebophila also resembles C. trachomatis in lacking the orthologs of enzymes for the lower DAP pathway other than dapF.

### DISCUSSION

The AT pathway of m-DAP/lysine synthesis extends to more bacterial genera than just Chlamydia and Protochlamydia. Corynebacterium glutamicum, a gram-positive soil bacterium, possesses both a succinylase and a dehydrogenase variant of the m-DAP/lysine pathway. C. glutamicum mutants carrying deletions in dapC, ddh and argD are still viable suggesting that this organism utilizes an unidentified mechanism to synthesize m-DAP (10). By BLAST alignment, CT390 shares homology with numerous C. glutamicum ATs as well as an annotated cystathionine β-lyase.

Chlamydiae cause significant disease worldwide in both humans and animals. C. trachomatis is the most prevalent cause of bacterial sexually transmitted infections as well as the leading cause of preventable infectious blindness. C. pneumoniae infections have been associated with coronary heart disease and atherosclerosis. Chlamydophila spp. are responsible for a wide variety of clinically and economically important diseases in poultry and livestock. Because m-DAP/lysine synthesis is unique to plants and bacteria, compounds that target this pathway are attractive candidates as herbicides and antimicrobials. Furthermore, inhibitors that directly target LL-DAP-ATs could have applications as chlamydiae-specific antibiotics.

### REFERENCES FOR EXAMPLE II

1. van Heijenoort, J. (2001) Nat Prod Rep 18, 503-19.
2. Harb, O, S. & Abu Kwaik, Y. (1998) Infect Immun 66, 1898-1903.
3. Burns-Keliher, L. L., Portteus, A. & Curtiss III, R. (1997) J Bacteriol 179, 3604-3612.
4. Cersini, A., Salvia, A. M. & Bernardini, M. L. (1998) Infect Immun 66, 549-557.
5. Hutton, C. A., Southwood, T. J. & Turner, J. J. (2003) Mini Rev Med Chem 3, 115-127.
6. Cox, R. J., Sutherland, A. & Vederas, J. C. (2000) Bioorg Med Chem 8, 843-871.
7. Velasco, A. M., Leguina, J. I. & Lazcano, A. (2002) J Mol Evol 55, 445-459.
8. Bukhari, A. I. & Taylor, A. L. (1971) J Bacteriol 105, 844-854.
9. Fuchs, T. M., Schneider, B., Krumbach, K., Eggeling, L. & Gross, R. (2000) J Bacteriol 182, 3626-3631.
10. Hartmann, M., Tauch, A., Eggeling, L., Bathe, B., Mockel, B., Puhler, A. & Kalinowski, J. (2003) J Biotechnol 104, 199-211.
11. Cox, R. J. & Wang, P. S. H. (2001) J Chem Soc, Perkin Trans 1, 2006-2008.
12. Weinberger, S. & Gilvarg, C. (1970) J Bacteriol 101, 323-324.
13. McCoy, A. J. & Maurelli, A. T. (2006) Trends Microbiol 14, 70-77.
14. Moulder, J. W., Novosel, D. L. & Tribby, I. C. (1963) J Bacteriol 85, 701-706.
15. Hudson, A. O., Singh, B. K., Leustek, T. & Gilvarg, C. (2006) Plant Physiol 140, 292-301.

### Example III

### Phylogenic Occurrence of the LL-DAP Aminotransferase

In order to assess how widely LL-diaminopimelate transaminase is distributed the sequenced microbial genomes were surveyed for orthologs and candidates were identified in both the eubacteria and archaea. Protein sequence comparison revealed that LL-diaminopimelate transaminase orthologs clustered into two divergent groups sharing approximately 30 to 60% identity. Functional analysis of selected representative enzymes showed that both groups contain authentic LL-diaminopimelate transaminases. The kinetic properties of these enzymes strongly supported the hypothesis that they function in the biosynthesis of diaminopimelate/lysine in that they were shown to be capable of synthesizing LL-diaminopimelate from tetrahydrodipicolinate in vitro and they were also able to complement an E. coli dapD/dapE mutant. The microbial species whose genomes have been sequenced were surveyed for the presence of other DAP pathway enzymes. In the majority of cases, LL-diaminopimelate transaminase was not coincident with the presence of DapD, DapC, or DapE, further supporting the idea that these species contain a novel variant of the DAP/lysine pathway. The LL-diaminopimelate transaminase pathway showed a limited phylogenetic distribution, being found in a few eubacterial groups including the Cyanobacteria, Desulfuromonadales, Firmicutes, Bacteroidetes, Chlamydiae, Spirochaeta; and a single archaeal group the Methanobacteriaceae.

The meso-diaminopimelate (m-DAP) is the immediate precursor of lysine in prokaryotes and plants (Bryan, 1990; Patte, 1996). In the eubacteria, m-DAP also serves a critical role in the synthesis of murein (von Heijenoort, 1996). The m-DAP pathway is one of the two lysine biosynthesis pathways to have evolved (Vogel, 1965). The other, which shares an evolutionary origin with the pathway for leucine biosynthesis (Velasco et al., 2002) utilizes the intermediate compound α-amino adipic acid (AAA). The AAA pathway is found in most fungi (Velasco et al., 2002) and a variant of it is found in selected eubacterial and archaeal species (Nishida et al., 1999). By contrast, the m-DAP pathway shares an evolutionary relationship with arginine biosynthesis.

The most recent DAP pathway to have been discovered uses two enzymes to convert THDPA to m-DAP. See Example I. The distinguishing enzyme of this pathway catalyzes the glutamate-dependent transamination of THDPA to form LL-DAP. m-DAP is then formed by epimerization. Based upon its constrained substrate specificity LL-DAP transaminase does not appear to be closely related to the DapC transaminase that functions in the acyl-DAP pathways. In fact, at least 2 different enzymes are known to catalyze the DapC reaction (Ledwidge and Blanchard, 1999; Fuchs et al., 2000; Cox and Wang, 2001; Hartmann et al., 2003). The LL-DAP transaminase has been reported in plants where it appears to be the only route for lysine biosynthesis. This assumption is based on the absence of orthologs for the acyl and Ddh pathway enzymes in the Arabidopsis thaliana genome and the absence of acyl pathway enzyme activities in several different plant species (Hudson et al., 2005).

The catalytic properties of the LL-DAP transaminase from A. thaliana is described in Example I and in (Hudson et al., 2006). It falls into the class 1, 2 superfamily of transaminases. The activity was found in a variety of plant species, but was not detected in a variety of bacterial species known to contain at least one of the other DAP pathway variants. The A. thaliana enzyme catalyzes a reversible reaction. The biosynthetic activity is disfavored over the reverse activity by a factor of approximately 60. It was hypothesized that the higher concentration of substrates over products drives the biosynthetic reaction in vivo. In the reverse direction the enzyme shows exquisite substrate specificity, being able to distinguish LL-DAP from its isomer m-DAP.

The presence of a fourth variant of the DAP pathway in plants raised the question of whether this variant exists in prokaryotes. If so, the additional examples of LL-DAP aminotransferase could shed light on the evolution of the DAP pathway. The present study made use of the extensive list of sequenced microbial genomes to search for orthologs of LL-DAP transaminase to provide targets for the identification of agents which inhibit bacterial growth.

The following materials and methods are provided to facilitate the practice of Example III.

### Bioinformatic Methods

Orthologous sequences were identified using blastp of the sequenced microbial genomes. Phylogenetic analysis was carried out using ClustalW (Thompson et al., 1994) and the neighbor-joining tree was constructed using MEGA 3.1 (Kumar et al., 2004). Genomic context was explored and DAP/lysine biosynthesis genes cataloged using the IMG database (Markowitz et al., 2006).

### Cloning

TABLE 1
Loci amplified for this study

| | | | Source of strain |
|---|---|---|---|
| | | | or genomic |
| Species | Locus | Amplification Primers | DNA |
| Bacteroides fragilis | BF2666 | 5′-GGGGGAATTCATGGCATTAGTAAACGAACATTTT | Sheila Patrick, |
| NCTC9343 | | TTGAAATTACC-3′ | Queen's |
| | | 5′-GGGGGTCGACTTACAGTCGGTTCTTGATGCGTCG | University of |
| | | CATCGCT-3′ | Belfast |
| Desulfitobacterium | DhaFDRAFT_3980 | 5′-GGGGCCATGGATGGCTCAGATCAATGAGAATTA | James M. Tiedje, |
| hafniense DCB-2 | | CT3′ | Michigan State |
| | | 5′-GGGGAAGCTTTTACTTCATCCGAGCTTTAATGCG | University |
| | | CT-3′ | |
| Gloeobacter violaceus | glr4108 | 5′-GGGGCCATGGATGAAGACTGCCGCCCGCCTCGAT | Takakazu |
| PCC 7421 | CGCAT-3′ | Kaneko, Kazusa | |
| | | 5′-GGGGGTCGACCTAGCCCTCGAAGCGGATCCCGG | DNA Research |
| | | CGGCCTC-3′ | Institute |
| Leptospira interrogans | LICI2841 | 5-′GGGGCCATGGCGAACATCAATGAAAATT-3′ | Albert Ko, Weill |
| serovar Copenhageni | | 5′-GGGGGTCGACTTAAAGAGATGTAATCCTTGCGA-3′ | Medical College |
| strain Fiocruz L1-130 | | of Cornell | |
| | | | University |
| Methanospirillum | Mhun_2943 | 5′-GGGGCCATGGATGTTTGCACAACGAATAGCGAAT | Caroline Plugge, |
| hungatei JF-1 | CTTCCCCCCT-3′ | Wegeningen | |
| | | 5′-GGGGGTCGACTCACCATTTCACCTCGCCCATCCGTT | University |
| | | | CTACTGCC-3′ |
| Methanothermobacter | MTH52 | 5′-GGGGGGATCCATGGTGACTGTAAACGAGAAC-3′ | John N. Reeve, |
| thermautotrophicus | | 5′-GGGGGTCGACCTAGAAGCTCAGCTCTGATAT-3′ | Ohio State |
| DeltaH | | | University |
| Moorella thermoacetica | Moth_0889 | 5′-GGGGCCATGGATGCAGGAAGCCAGAAGGATTCG | Stephen W. |
| ATCC 39073 | | CGAACT-3′ | Ragsdale, |
| | | 5′-GGGGAAGCTTTTAAAATTCGACCTTCCCCAGGACC | University of |
| | | CG-3′ | Nebraska |
| Synechocystis sp. PC | sll0480 | 5′-GGGGGGATCCATGGCCAGTATCAACGACAAC-3′ | ATCC #27184 |
| 6803 | | 5′-GGGGGTCGACCTAACCCAATTTGAGGGTGGA-3′ | |
| Syntrophobacter | SfumDRAFT_0821 | 5′-GGGGGAATTCATGGCATTCGTCAAAGCGGAACGG-3′ | Caroline Plugge, |
| fumaroxidans MPOB | | 5′-GGGGGTCGACTCAAAAAGACAACTTCCGCATCCG | Wegeningen |
| | | TTCG-3′ | University |
| | | | (Harmsen et al., 1998) |
| Parachlamydia | pc0685 | 5′-GGGGCCATGGAACTTCTTCCTCAAGAGTGGA-3′ | Matthias |
| | | 5′-GGGGGTCGACTCAGTAAACTGTAGGCTTAAT-3′ | Horn |
| Chlamydia trachomatis | Ct390 | 5′-GGGGCCATGGATGAAAAGAAACCCTCACTTCGTATCAT-3′ | Anthony |
| | | 5′-GGGGGTCGACTTATGATTGCAGAGCAGGAGCCAT-3′ | Maaurelli |

Methanospirillum hungatei JF-1, Synechocystis sp. PC 6803, and Syntrophobacter fumaroxidans MPOB were provided as cells from which genomic DNA was isolated. For all others pure genomic DNA was provided.

All orfs were processed identically for expression in E. coli. The orfs were amplified by PCR using the indicated primers. The resulting DNA fragment was digested with BamHI and SalI and cloned into pET30b. The pET30b clone was sequenced. This plasmid was transformed into E. coli BL21-CodonPlus®-RIPL for expression and purification of the recombinant protein. For functional complementation experiments, the expression cassette was subcloned from pET30b into pBAD33 (Guzman et al., 1995) using XbaI and SalI. Using these enzymes the expression cassette included the entire orf, the His-Tag coding sequence and the ribosome binding site from pET30b. pBAD33 provided an arabinose-regulated promoter.

### Protein Expression and Purification

For protein expression the E. coli strains were grown on LB at 37° C. to an OD600 nm of 0.5 and protein expression was then induced with 1 mM IPTG for 4 hr at 25° C. Cells were lysed by sonication in a solution of 50 mM sodium phosphate and 300 mM NaCl (pH 8.0). Metabolites in the extract were removed by buffer exchange using an Amicon Ultra 30,000 MWCO ultrafilter and sodium phosphate/NaCl buffer, and the concentrated soluble protein sample was used for measurement of enzyme activity as an initial assessment of enzyme function. When the recombinant protein was to be purified a larger cell culture was grown and lysed as described above, but the buffer exchange step was not carried out. Rather, the soluble protein was incubated with Talon metal affinity agarose (Clontech #8901-2), which was then washed 3 times with sodium phosphate/NaCl buffer containing 10 mM imidazole and finally the bound protein was eluted with sodium phosphate/NaCl buffer containing 300 mM imidazole. The pure protein was then concentrated in an Amicon Ultra 30,000 MWCO ultrafilter, replacing the elution buffer with 100 mM HepesKOH, pH 7.6.

### Functional Complementation and Enzyme Assays

In functional complementation E. coli strain AOH1, a dapD and dapE mutant (Hudson et al., 2006) was transformed with either the plasmid vector or with LL-DAP-AT expression plasmids. Transformants were selected on LB medium supplemented with 50 μg mL⁻¹ DAP (DL-α,ε-diaminopimelic acid, Sigma-Aldrich product #D-1377) and 34 μg mL⁻¹ chloramphenicol. Individual colonies were then replica plated onto NZY medium supplemented with 0.2% (w/v) arabinose without or with 50 μg mL⁻¹ DAP. The cultures were grown at 30° C. for 48 h.

### Enzyme Assays

Enzyme assays were performed as described above in Examples I and II.

### Results

### Identification of LL-DAP Transaminase Orthologs in Microbial Genomes

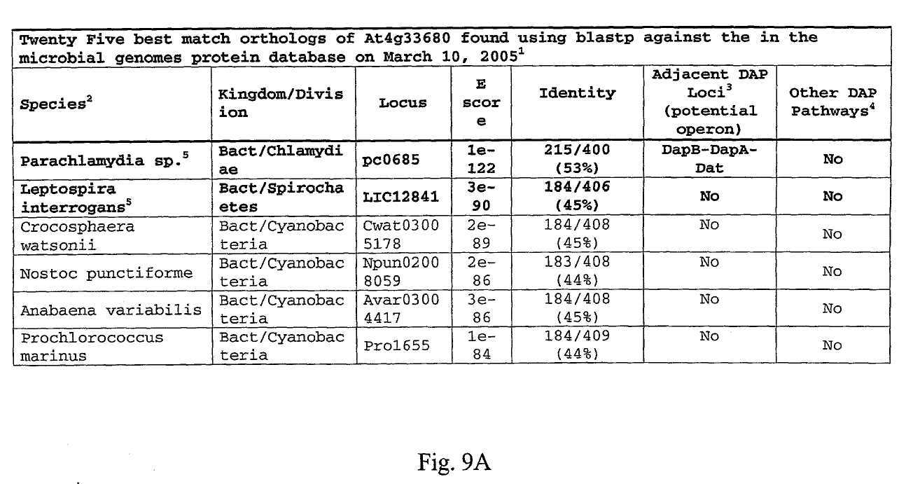

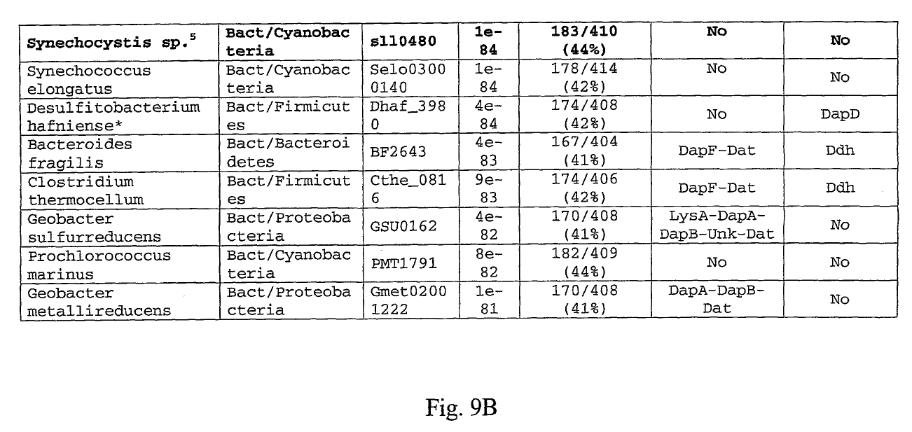

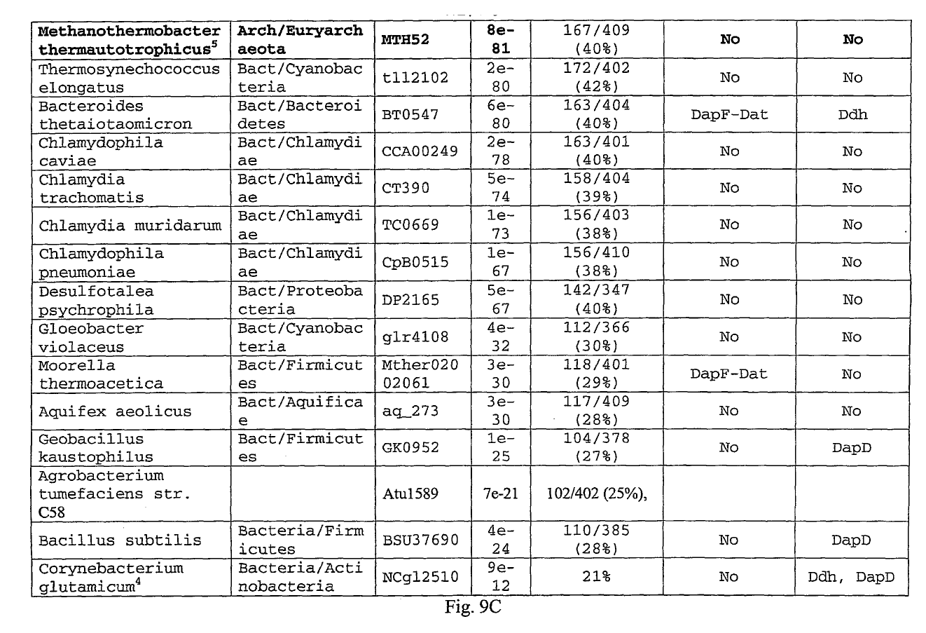

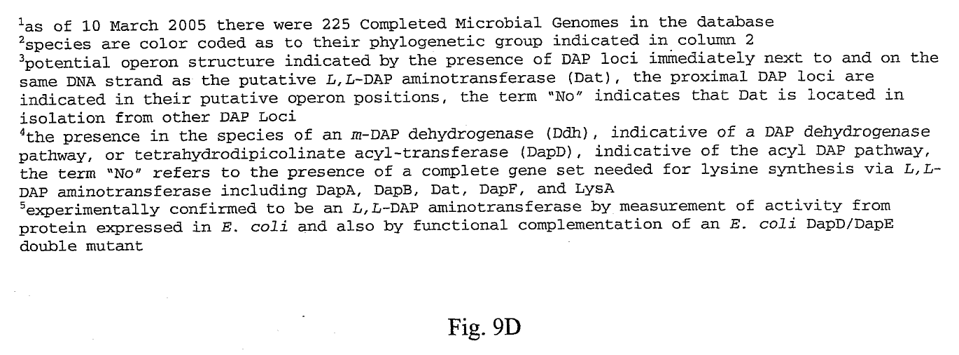

LL-DAP transaminase from A. thaliana was identified as the product of locus At4g33680 (Example I). The mature A. thaliana LL-DAP transaminase is 402 amino acids and contains a sequence motif that defines it as a member of the protein superfamily of Class 1,2 transaminases (Sung et al., 1991; Jensen and Gu, 1996). The At4g33680 sequence was used as the query to search for orthologs in the NCBI microbial genomes protein sequence database using blastp. The best match was with a protein derived from Candidatus Protochlamydia amoebophila, locus pc0685 with which it showed 56% identity (FIG. 9). Included among the best matches were sequences from only a few eubacterial groups including the Cyanobacteria, Desulfuromonadales, Firmicutes, Bacteroidetes, Chlamydiae, and Spirochaeta. In addition, several sequences from archaeal species were found, all of them from members of the Methanobacteriaceae.

It was interesting to note that the best matches to At4g33680 included proteins from all the cyanobacterial species whose genomes have been sequenced. The likely ancestor of chloroplasts is thought to have been a cyanobacteria. The divergence in sequence homology between the cyanobacterial representatives and At4g33680 was significant, however. The best matches ranged from 42% to 45% identity with At4g33680. The most diverged sequence was glr4108 from Gloeobacter violaceus, which showed only 30% identity with At4g33680. The low overall homology raised the question of the level of sequence identity that might define an authentic LL-DAP transaminase. An approximate lower limit could be estimated at about 28% identity based on the observation that this level of homology was observed with the loci BSU37690 and Atu1589 from Bacillus subtilis and Agrobacterium tumefaciens, species that were previously examined and found to be devoid of LL-DAP transaminase activity (Hudson et al., 2006). Thus, BSU37690 and Atu1589 are not likely to encode LL-DAP transaminase. The estimated lower homology limit was also supported by the observation that transaminase paralogs often show approximately 28% identity. A typical example is the case of Synechocystis sp. in which, the closest paralog of sll0480 (the locus identified as the LL-DAP transaminase) is sll0938 with which it is 26.6% identical.

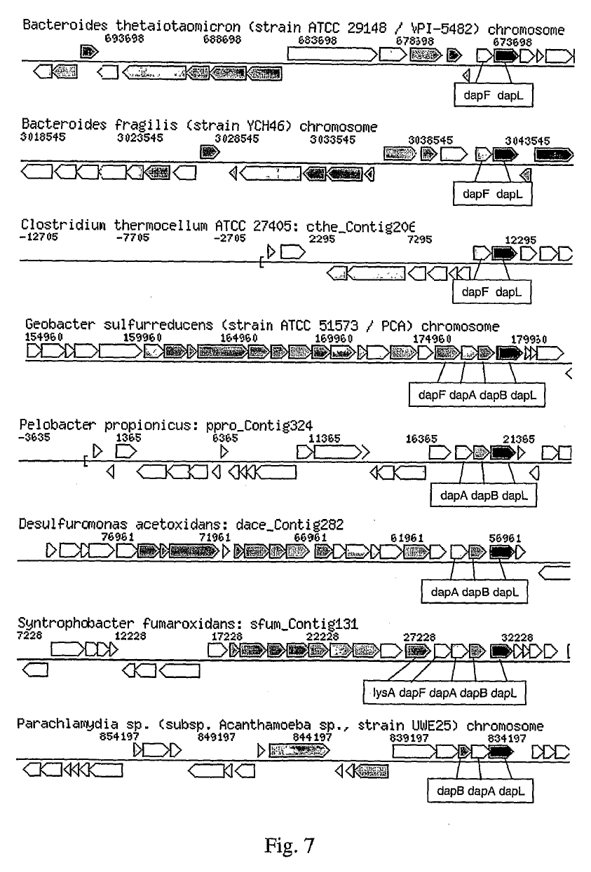

Considering the overall low homology of the best match sequences with At4g33680, further evidence for function was sought by examining the genomic context of the microbial LL-DAP transaminase orthologs. Genetic linkage to other m-DAP/lysine biosynthesis genes would further suggest a functional role for these loci. Although none of the cyanobacterial LL-DAP transaminase orthologs were found in association with other DAP/lysine biosynthesis genes, in several other cases a strong genetic linkage was observed. See FIG. 7. In several of the Firmicutes and the Bacteroidetes the orthologs were found to lie immediately downstream of and on the same DNA strand as dapF. In Parachlamydia sp. the LL-DAP transaminase ortholog was found downstream of and on the same strand as dapB and dapA. In the Desulfuromonadales the LL-DAP transaminase orthologs were found to lie immediately downstream of and on the same DNA strand as dapA and dapB. Geobacter sulfurreducens contains in addition, the lysA gene within the cluster. An extreme case exists for Syntrophobacter fumaroxidans, in which the genomic context also includes lysA and dapF. Thus, if S. fumaroxidans SfumDRAFT_{—}0821 does indeed encode LL-DAP transaminase the entire lysine biosynthesis pathway would be encoded at one locus in this species, perhaps within a single transcriptional unit.

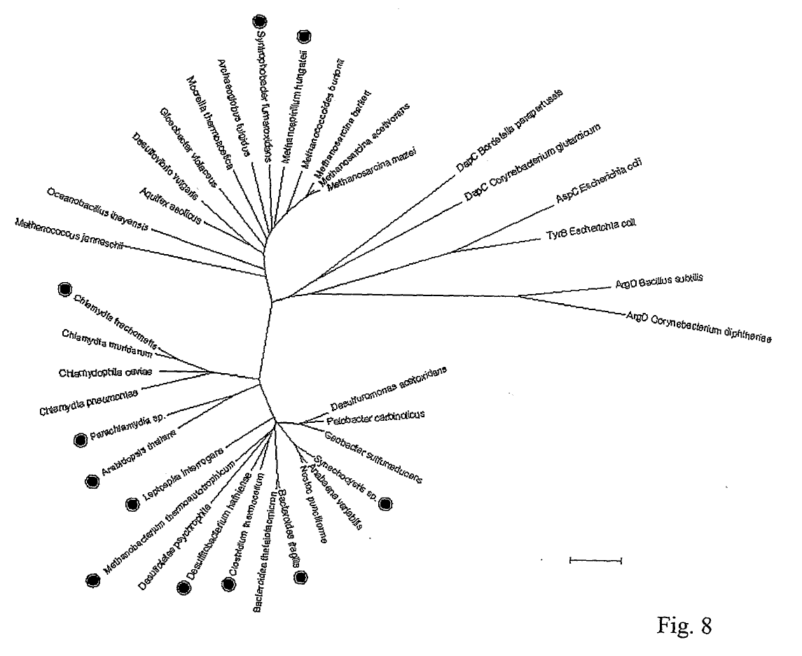

Despite the suggestive genomic context for S. fumaroxidans SfunDRAFT_{—}0821, this protein showed only slightly greater homology with A. thaliana LL-DAP transaminase than did BSU37690 and Atu1589, two proteins that were deemed to be unlikely LL-DAP transaminase candidates. This suggested that LL-DAP transaminases might be highly diverged. To assess the extent of the divergence the collection of microbial LL-DAP transaminase orthologs was used to create the neighbor-joining tree shown in FIG. 8. Also included in the tree for the sake of comparison were two DapC and two ArgD sequences. These were included because they were previously demonstrated to catalyze aminotransfer to N-succinyl-L-2-amino-6-oxopimelate (Ledwidge and Blanchard, 1999; Fuchs et al., 2000; Hartmann et al., 2003), which is the reaction in the acyl DAP pathways analogous to that catalyzed by LL-DAP transaminase. Additionally, E. coli AspC and TyrB were included because the LL-DAP transaminases identified in FIG. 9 were most often annotated as aspartate aminotransferase or aromatic amino acid transaminase. The phylogenetic analysis showed that LL-DAP transaminase orthologs cluster into two divergent groups, both of which are distantly related to DapC, ArgD, AspC or TyrB. The cluster containing A. thaliana At4g33680 includes sequences that are primarily derived from eubacteria, the single exception being MTH52 from the archaeal species Methanothermobacter thermoautotrophicum. This cluster is further divided into distinct lineages for the Cyanobacteria, Desulfuromonadales, Firmicutes, Bacteroidetes, and Chlamydiae (FIG. 8). The second major cluster includes many archaeal species and several eubacteria including S. fumaroxidans SfumDRAFT_{—}0821. One of the archaeal sequences, Mhun_{—}2943 from Methanospirillum hungatei is of particular of interest because this species is the syntrophic partner of S. fumaroxidans. Also of interest is the presence in this cluster of glr4108, the most divergent cyanobacterial LL-DAP transaminase ortholog.

### Functional Analysis of Microbial LL-DAP Transaminases

To assess the function of enzymes from each of the clusters illustrated in FIG. 7, eight different proteins were selected for expression in E. coli and examination of their catalytic properties. They included sll0480 from Synechocystis sp., BF₂₆₄₃ from Bacteroides fragilis, Dhaf_{—}3980 from Desulfitbacterium hafniense, MTH52 from M. thermoautotrophicum, LIC12841 from Leptospira interrogans, SfumDRAFT0821 from S. fumaroxidans, Mhun_{—}2943 from Methanospirillum hungateii, and Mther02002061 from Moorella thermoacetica. As a basis for comparison the catalytic properties for each of these enzymes was compared with that of A. thaliana At4g33680, which was previously reported (Hudson et al., 2006). A convenient assay for the activity of LL-DAP transaminase is its ability to bypass the dapD, dapC, and dapE reactions when expressed in E. coli. The dapD and dapE mutants are auxotrophic for DAP and complementation can be easily assessed as restoration of the strain to DAP prototrophy. Both mutations were combined into one strain in order to be certain that complementation bridges the entire metabolic gap from THDPA to m-DAP. FIG. 8 shows that all the orthologs were able to complement the E. coli dapD/dapE mutant. Moreover, complementation was dependent on the presence of arabinose in the medium, as was expected given that the orf's were cloned under control of an arabinose-activated promoter. These results indicate that the all the microbial enzymes tested are indeed LL-DAP transaminases. Moreover, the complementation results further indicated that the enzymes are able to function in the biosynthetic direction under physiological conditions.

The complementation result was further explored by examining the specificity of the transaminases for LL-DAP compared with m-DAP, the activity was measured from soluble protein extracts of the E. coli strains expressing the heterologous prokaryotic enzymes or directly from the microbial host species. The measurements revealed that all the enzymes tested showed LL-DAP transaminase activity and that all showed exquisite substrate specificity as described in Table 1 for the LL-DAP-AT from Arabidopsis.

The catalytic properties of sll0480, BF2643, Dhaf_{—}3980, MTH52, and SfumDRAFT0821 were studied in greater detail using purified enzymes to determine their kinetic constants using quantitative forward and reverse assays. Biosynthetic forward activity was measured by coupling the production of 2-oxoglutarate to NAD⁺

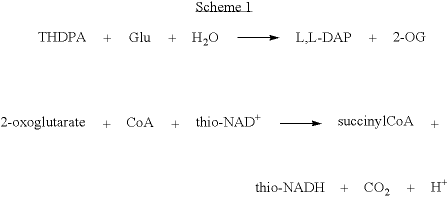

Reverse activity was assayed by coupling THDPA formation to NADPH oxidation using DAP dehydrogenase as depicted in Scheme 2.

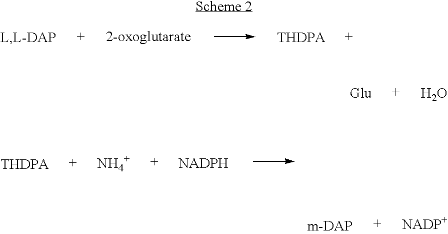

| | Vmax F | Vmax R | Km | Km | Km | |
|---|---|---|---|---|---|---|
| | (micromol/ | (micromol/ | 2-OG | L,L-DAP | THDPA | Km glu |
| Enzyme | min/mg) | min/mg) | (mM) | (mM) | (mM) | (mM) |
| sII0480 | 0.25 | 3.3 | 0.31 | 0.017 | 0.013 | 1.5 |
| Mth52 | 0.6 | 4.7 | 0.8 | 0.03 | 0.075 | 3.0 |
| Dhaf_3890 | 0.3 | 4 | 0.3 | 0.041 | 0.07 | 0.8 |
| SfumDRAFT_0821 | 0.26 | 3.4 | 0.7 | 0.029 | 0.012 | 0.7 |
| Bf2643 | 0.3 | 3.6 | 1.2 | 0.01 | 0.096 | 1.3 |
| At4g33680 | 0.8 | 12 | 1.4 | 0.062 | 0.025 | 1.4 |

Some of the loci that have here been identified as LL-DAP transaminases were initially annotated as aromatic amino acid transaminases, or aspartate transaminases, or alanine transaminases. Whether the enzymes display these activities was determined by substituting a variety of keto acids for 2-oxoglutarate in the reverse reaction and for THDPA in the forward reaction. sll0480 was active only with 2-oxoglutarate or THDPA. There was no activity even with very high concentrations (5 mM) of the substitute keto acids. This result supports the hypothesis that LL-DAP transaminases are highly specific enzymes that function in DAP/lysine biosynthesis.

### Phylogenetic Distribution of Variant DAP Pathways

Given the unusual natural diversity observed in the DAP/lysine pathway it was of interest to determine the phylogenetic distribution of the pathway variants. This effort could shed light on the evolution of this pathway and whether coincidence of two DAP pathways, such as that found in C. glutamicum, is common. This effort is currently possible as a result of the large number of microbial species whose genome sequences have been sequenced. At present, the genomes of 424 species and strains have been completely sequenced or are in various stages of completion. These genomes were searched for orthologs of DapA, DapB and LysA as representative of the common steps of the DAP pathway variants. They were also searched for DapD and DapE, the two genes that represent the acyl pathway variants, and DapF, which is found in both the acyl and LL-DAP transaminase variants. DapC was not examined because there are at least two gene products.

### Discussion

The net reaction for this conversion is the transfer of a single amino group to THDPA to form LL-DAP followed by an isomerization to m-DAP. However, the THDPA molecule is cyclic and lacks the keto group needed for transamination. It exists in solution along with its acyclic form L-2-amino-6-oxopimelate, which carries a keto group. The equilibrium favors THDPA. In some species, ring-opening is catalyzed by N-succinylation or N-acetylation of THDPA using succinyl-CoA or acetyl-CoA as an acyl-group donor. The exposed keto group then serves as the amino acceptor for a transamination reaction. Finally, the succinyl or acetyl group is hydrolyzed to form LL-DAP. The enzymes catalyzing these reactions in E. coli are DapD, DapC, and DapE. Although the function of acylation has never been clearly established it has been proposed to speed the rate of opening of the THDPA ring. Both pathway variants are widely distributed in the eubacteria and archaea.

### REFERENCES FOR EXAMPLE III

Bryan J K (1990) Advances in the biochemistry of amino acid biosynthesis. In B J Miflin, P J Lea, eds, The biochemistry of plants, Vol 16. Academic Press, New York, pp 161-195
Cox R J, Wang P S H (2001) Is N-acetylomithine aminotransferase the real N-succinyl-LLdiaminopimelate aminotransferase in Escherichia coli and Mycobacterium smegmatis? J. Chem. Soc. Perkin Trans. 1: 2006-2008
Fuchs T M, Schneider B, Krumbach K, Eggeling L, Gross R (2000) Characterization of a Bordetella pertussis diaminopimelate (DAP) biosynthesis locus identifies dapC, a novel gene coding for an N-succinyl-L,L-DAP aminotransferase. J Bacteriol 182: 3626-3631
Gilvarg C (1959) N-Succinyl-L-diaminopimelic acid. J Biol Chem 234: 2955-2959
Gilvarg C (1961) N-Succinyl-alpha-amino-6-ketopimelic acid. J Biol Chem 236: 1429-1431
Guzman L M, Belin D, Carson M J, Beckwith J (1995) Tight regulation, modulation, and high-level expression by vectors containing the arabinose PBAD promoter. J Bacteriol 177: 4121-4130
Harmsen H J, Van Kuijk B L, Plugge C M, Akkermans A D, De Vos W M, Stams A J (1998) Syntrophobacter funaroxidans sp. nov., a syntrophic propionate-degrading sulfate-reducing bacterium. Int J Syst Bacteriol 48 Pt 4: 1383-1387
Hartmann M, Tauch A, Eggeling L, Bathe B, Mockel B, Puhler A, Kalinowski J (2003) Identification and characterization of the last two unknown genes, dapC and dapF, in the succinylase branch of the L-lysine biosynthesis of Corynebacterium glutamicum. J Biotechnol 104: 199-211
Hudson A O, Bless C, Macedo P, Chatterjee S P, Singh B K, Gilvarg C, Leustek T (2005) Biosynthesis of lysine in plants: evidence for a variant of the known bacterial pathways. Biochim Biophys Acta 1721: 27-36
Hudson A O, Singh B K, Leustek T, Gilvarg C (2006) An LL-diaminopimelate aminotransferase defines a novel variant of the lysine biosynthesis pathway in plants. Plant Physiol 140: 292-301
Jensen R A, Gu W (1996) Evolutionary recruitment of biochemically specialized subdivisions of Family I within the protein superfamily of aminotransferases. J Bacteriol 178: 2161-2171
Kumar S, Tamura K, Nei M (2004) MEGA3: Integrated software for Molecular Evolutionary Genetics Analysis and sequence alignment. Brief Bioinform 5: 150-163
Ledwidge R, Blanchard J S (1999) The dual biosynthetic capability of N-acetylomithine aminotransferase in arginine and lysine biosynthesis. Biochemistry 38: 3019-3024
Markowitz V M, Korzeniewski F, Palaniappan K, Szeto E, Werner G, Padki A, Zhao X, Dubehak I, Hugenholtz P, Anderson I, Lykidis A, Mavromatis K, Ivanova N, Kyrpides N C (2006) The integrated microbial genomes (IMG) system. Nucleic Acids Res 34: D344-348
Misono H, Togawa H, Yamamoto T, Soda K (1976) Occurrence of meso-alpha, epsilon-diaminopimelate dehydrogenase in Bacillus sphaericus. Biochem Biophys Res Commun 72: 89-93
Nishida H, Nishiyama M, Kobashi N, Kosuge T, Hoshino T, Yamane H (1999) A prokaryotic gene cluster involved in synthesis of lysine through the amino adipate pathway: a key to the evolution of amino acid biosynthesis. Genome Res 9: 1175-1183
Patte J-C (1996) Biosynthesis of threonine and lysine. In F C Neidhardt, ed, Escherichia coli and Salmonella Cellular and Molecular Biology, Ed Second Vol 1. ASM Press, Washington D.C., pp 528-541
Schrumpf B, Schwarzer A, Kalinowski J, Puhler A, Eggeling L, Sahm H (1991) A functionally split pathway for lysine synthesis in Corynebacterium glutamicium. J Bacteriol 173: 4510-4516
Sundharadas G, Gilvarg C (1967) Biosynthesis of alpha,epsilon-diaminopimelic acid in Bacillus megaterium. J Biol Chem 242: 3983-3984
Sung M H, Tanizawa K, Tanaka H, Kuramitsu S, Kagamiyama H, Hirotsu K, Okamoto A, Higuchi T, Soda K (1991) Thermostable aspartate aminotransferase from a thermophilic Bacillus species. Gene cloning, sequence determination, and preliminary x-ray characterization. J Biol Chem 266: 2567-2572
Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res 22: 4673-4680
Velasco A M, Leguina J I, Lazcano A (2002) Molecular evolution of the lysine biosynthetic pathways. J Mol Evol 55: 445-459
Vogel H J (1965) Lysine biosynthesis and evolution. In V Bryson, H J Vogel, eds, Evolving genes and proteins. Academic Press, New York, pp 25-40
von Heijenoort J (1996) Murein synthesis. In FC Neidhardt, ed, Escherichia coli and Salmonella Cellular and Molecular Biology, Ed Second Vol 1. ASM Press, Washington D.C., pp 1025-1034
Weinberger S, Gilvarg C (1970) Bacterial distribution of the use of succinyl and acetyl blocking groups in diaminopimelic acid biosynthesis. J Bacteriol 101: 323-324
White P J (1983) The essential role of diaminopimelate dehydrogenase in the biosynthesis of lysine by Bacillus sphaericus. J. Gen. Microbiol. 129: 739-749

### Example IV

### Alternative Methods for Enhancing Lysine Content in Higher Plants

Improvement of the nutritional value of crops is currently a major goal for agricultural companies. Fermentative production of lysine for sale as nutritional supplement is a major industry. Clearly, methods which are effective to increase the lysine content in cells from important crops are highly desirable. Thus, in yet another aspect of the invention, transgenic plants are provided wherein the LL-DAP-AT pathway is bypassed or augmented. This can be achieved by introducing exogenous nucleic acids encoding DAP dehydrogenase (representative amino acid sequences for DAP are provided in FIGS. 10A and 10B) into the cells of higher plants thereby increasing the conversion of tetrahydrodipicolinate to LL-diaminopimelate and increasing lysine biosynthesis in the cell. Plants regenerated from such plant cells are also encompassed by the present invention Such plants may optionally comprise a heterologous nucleic acid encoding the LL-DAP-AT enzyme described herein. Augmenting the lysine biosynthesis pathways in this way should increase the lysine content in the resulting plant cell, with crop plants being particularly preferred.

In yet another approach, a plurality of nucleic acids encoding the acylating diaminopimelate pathway are introduced into a plant cell. These enzymes include L-2,3,4,5-tetrahydrodipicolinate acyl-transferase, N-succinyl-L-diaminopimelic glutamic transaminase, and N-succinyl-L-alpha,epsilon-diaminopimelic acid deacylase, commonly referred to as DapD, DapC, and DapE, respectively. They are also identified by the Enzyme Commission nomenclature EC 2.3.1.117, EC 2.6.1.17 and EC 3.5.1.18, respectively. Representative amino acid sequences for these enzymes are provided in FIG. 11. Alternatively the acetylating DapD, DapC and Dap E enzymes may be employed. Provision of these enzymes should also effectively increase the lysine content in a plant cell. Plant cells containing nucleic acids expressing the foregoing enzymes may also comprise the LL-DAP-AT described herein. As above, transgenic plants regenerated from such plant cells also comprise an aspect of the present invention.

While certain preferred embodiments of the present invention have been described and specifically exemplified above, it is not intended that the invention be limited to such embodiments. Various modifications may be made to the invention without departing from the scope and spirit thereof as set forth in the following claims.

1. A nucleic acid molecule encoding an LL-DAP amino transferase.

2. The nucleic acid of claim 1, isolated from an organism selected from the group consisting of those shown in FIG. 9.

3. The nucleic acid of claim 1, isolated from Arabidopsis thaliana.

4. The nucleic acid of claim 1, isolated from a crop plant.

5. A vector comprising the aminotransferase-encoding nucleic acid of claim 1.

6. A transgenic cell comprising the vector of claim 5.

7. The cell of claim 6, selected from the group consisting of a pathogenic bacterial cell, a plant cell, an algae, and a chlamydial cell.

8. A transgenic plant regenerated from the plant cell of claim 7.

9. A method for identifying a compound which modulates LL-diaminopimelate aminotransferase activity comprising:
a) incubating said aminotransferase in the presence and absence of said compound under conditions which promote aminotransferase activity;
b) determining the amount of product formed in the presence and absence of said test compound, compounds which alter the amount of product formed having LL-diaminopimelate aminotransferase modulating activity.

10. The method of claim 9, wherein said aminotransferase catalyzes the interconversion of tetrahydrodipicolinate and LL-diaminopimelate.

11. The method of claim 9, wherein said test compound increases said product formation.

12. The method of claim 9, wherein said test compound reduces said product formation.

13. The method of claim 9, performed in vitro.

14. The method of claim 9 performed in vivo.

15. The method of claim 12, wherein said organism is an organism listed in FIG. 9.

16. A test compound identified by the method of claim 9.

17. The test compound of claim 16 which inhibits aminotransferase activity and is an herbicide

18. The test compound of claim 16 which inhibits aminotransferase activity and is an algaecide.

19. The test compound of claim 16 which inhibits aminotransferase activity and is an antibiotic or antibacterial agent

20. A test compound identified by the method of claim 11.

21. A method for enhancing lysine production in a higher plant comprising administering an effective amount of the test compound claim 11 to said plant.

22. The method of claim 13, wherein said test compound is administered to said plant via a method selected from the group consisting of spraying, watering, and application of soil pellets comprising said compound.

23. A method for enhancing lysine production in a cell comprising overexpressing an LL-diaminopimelate aminotransferase encoding nucleic acid of claim 1 in said cell.

24. The method of claim 23, wherein said cell is a plant cell.

25. The method of claim 24, wherein said plant cell is obtained from a crop plant selected from the group consisting of corn, sugarcane, beans, rice, wheat, oats, soybean, tobacco, sorghum, tomatoes, strawberries, parsley, sage, rosemary, and thyme.

6. The method of claim 25, wherein said amino transferase encoding nucleic acid further comprises sequences suitable for expression and production of said amino transferase in the plant plastid.

27. The method of claim 23, wherein said cell is a bacterial cell.

28. A method of enhancing the conversion of tetrahydrodipicolinate to L,L-diaminopimelate in a cell comprising:
a) introducing a nucleic acid encoding DAP dehydrogenase into a plant cell, or
b) introducing a plurality of nucleic acids encoding DapC, Dap D and Dap E into said cell.

29. (canceled)

30. A plant cell obtained from the method of claim 28.

31. A plant regenerated from the plant cell of claim 30.

32. The plant of claim 31, further comprising a heterologous nucleic acid encoding LL-DAP-AT.

33. A LL-DAP-AT produced by expression of the nucleic acid of claim 1.

34. A test compound identified by the method of claim 15, which inhibits growth of an organism listed in FIG. 9.


SEQUENCE LISTING (39 sequences)

SEQ ID NO: 1 - DNA, 21 bp, Artificial Sequence
  Synthetic Sequence
aatggagatcggccagaaaaa

SEQ ID NO: 2 - DNA, 21 bp, Artificial Sequence
  Synthetic Sequence
ggtgcccgaattacaaccatt

SEQ ID NO: 3 - DNA, 48 bp, Artificial Sequence
  Synthetic Sequence
ggggcattggaaggagatataaccatggcagtcaatacttgcaaatgt

SEQ ID NO: 4 - DNA, 37 bp, Artificial Sequence
  Synthetic Sequence
gggggtcgactcatttgtaaagctgcttgaatcttcg

SEQ ID NO: 5 - DNA, 31 bp, Artificial Sequence
  Synthetic Sequence
ggggggatccatggccagtatcaacgacaac

SEQ ID NO: 6 - DNA, 31 bp, Artificial Sequence
  Synthetic Sequence
gggggtcgacctaacccaatttgagggtgga

SEQ ID NO: 7 - protein, 461 aa, Arabidopsis thaliana [sequence omitted]

SEQ ID NO: 8 - DNA, 1587 bp, Arabidopsis thaliana
tttgctctgcaaatttgtctgaaaaaatctttgtgcgaagcaggaaaaatgtcgtcgacccatcagttagtttcttcgatgatctcttcttcctcatccactttcttagccccttcaaattttaatctcagaactcgaaatgcttgcttacccatggcaaaacgggtcaatacttgcaaatgtgttgctacgccgcaagagaagatcgagtataagaccaaagtgtcacggaattcaaacatgtccaaacttcaggctggatacctattcccggagattgcaagaagaaggtctgcacacttgttgaaatatccagatgcacaagttataagtcttggaataggcgacacaactgagccaattcctgaagtgatcacttctgctatggcaaagaaagctcatgagttgtcaacaatagagggatatagtggttatggtgctgaacaaggtgcaaagccactgagagctgctattgcgaaaacattctacggtggccttggcataggggatgatgacgtttttgtttctgatggagctaaatgtgatatctcacgtctccaggttatgtttggttccaatgttacaattgctgttcaggatccttcatatccggcttatgtggactccagtgttattatgggtcagactgggcaatttaacactgatgtgcaaaagtatggaaacatcgagtacatgagatgcactccagagaatggcttctttcccgacttgtccaccgttggcaggacagatataattttcttctgttccccaaataaccctacgggtgctgctgccacgagagagcaactaacgcagttagttgagtttgcaaagaagaacggttctataatagtgtatgattccgcctatgcaatgtacatgtctgatgataacccacgatccatcttcgaaatccctggagcagaggaggtcgctatggagacagcttcgttcagcaaatatgctggtttcactggagttcgacttggttggactgtcatcccgaaaaagctactctattcagacggtttccctgttgccaaagacttcaatcggattatctgcacttgtttcaatggtgcatctaatatctctcaagctggtgctcttgcttgccttacacccgaaggacttgaggcaatgcataaggtgattggattctataaagaaaacacaaacataatcattgacacattcacatctctcgggtatgatgtatatggaggaaagaatgcgccttacgtatgggttcacttcccgaaccaaagctcatgggatgtgtttgctgagattctggagaagactcatgtggttacaactccaggaagtgggtttggaccagggggtgaagggttcgttcgtgtcagtgcctttggtcacagagagaacatcttagaggcatgtcgaagattcaagcagctttacaaatgaagaaccttgtttgtaatcgttcctcatcatcatcaccctctttaatgacatgatttgagttaaaataatgtcgtttccattgttttctggaatttgtagaagacacttttgacaccagtgtttcaagcaatttggcaaagtattttaacaacg

SEQ ID NO: 9 - protein, 394 aa, Chlamydia trachomatis
MKRNPHFVSLTKNYLFADLQKRVAQFRLENPQHTVINLSIGDTTQPLNASVAEAFASSIARLSSPTTCRGYGPDFGLPALRQKLSEDFYRGFVDAKEIFISDGAKVDLFRLLSFFGPNQTVAIQDPSYPAYLDIARLTGAKEIIALPCLQENAFFPEFPEDTHIDILCLCSPNNPTGTVLNKDQLRAIVHYAIEHEILILFDAAYSTFISDPSLPKSIFEIPDARFCAIEINSFSKPLGFAGIRLGWTVIPQELTYADGHFVIQDWERFLSTTFNGASIPAQEAGVAGLSILPQLEAIHYYRENSDLLRKALLATGFEVFGGEHAPYLWVKPTQANISDRDLFDFFLREYHIAITPGIGFGRSGSGFVRFSSLGKREDILAACERLQMAPALQS

SEQ ID NO: 10 - DNA, 45 bp, Artificial Sequence
  Synthetic Sequence
gggggaattcatggcattagtaaacgaacattttttgaaattacc

SEQ ID NO: 11 - DNA, 41 bp, Artificial Sequence
  Synthetic Sequence
gggggtcgacttacagtcggttcttgatgcgtcgcatcgct

SEQ ID NO: 12 - DNA, 35 bp, Artificial Sequence
  Synthetic Sequence
ggggccatggatggctcagatcaatgagaattact

SEQ ID NO: 13 - DNA, 36 bp, Artificial Sequence
  Synthetic Sequence
ggggaagcttttacttcatccgagctttaatgcgct

SEQ ID NO: 14 - DNA, 39 bp, Artificial Sequence
  Synthetic Sequence
ggggccatggatgaagactgccgcccgcctcgatcgcat

SEQ ID NO: 15 - DNA, 40 bp, Artificial Sequence
  Synthetic Sequence
gggggtcgacctagccctcgaagcggatcccggcggcctc

SEQ ID NO: 16 - DNA, 28 bp, Artificial Sequence
  Synthetic Sequence
ggggccatggcgaacatcaatgaaaatt

SEQ ID NO: 17 - DNA, 33 bp, Artificial Sequence
  Synthetic Sequence
gggggtcgacttaaagagatgtaatccttgcga

SEQ ID NO: 18 - DNA, 44 bp, Artificial Sequence
  Synthetic Sequence
ggggccatggatgtttgcacaacgaatagcgaatcttcccccct

SEQ ID NO: 19 - DNA, 44 bp, Artificial Sequence
  Synthetic Sequence
gggggtcgactcaccatttcacctcgcccatccgttctactgcc

SEQ ID NO: 20 - DNA, 31 bp, Artificial Sequence
  Synthetic Sequence
ggggggatccatggtgactgtaaacgagaac

SEQ ID NO: 21 - DNA, 31 bp, Artificial Sequence
  Synthetic Sequence
gggggtcgacctagaagctcagctctgatat

SEQ ID NO: 22 - DNA, 39 bp, Artificial Sequence
  Synthetic Sequence
ggggccatggatgcaggaagccagaaggattcgcgaact

SEQ ID NO: 23 - DNA, 37 bp, Artificial Sequence
  Synthetic Sequence
ggggaagcttttaaaattcgaccttccccaggacccg

SEQ ID NO: 24 - DNA, 31 bp, Artificial Sequence; identical to SEQ ID NO: 5
  Synthetic Sequence

SEQ ID NO: 25 - DNA, 31 bp, Artificial Sequence; identical to SEQ ID NO: 6
  Synthetic Sequence

SEQ ID NO: 26 - DNA, 34 bp, Artificial Sequence
  Synthetic Sequence
gggggaattcatggcattcgtcaaagcggaacgg

SEQ ID NO: 27 - DNA, 38 bp, Artificial Sequence
  Synthetic Sequence
gggggtcgactcaaaaagacaacttccgcatccgttcg

SEQ ID NO: 28 - DNA, 31 bp, Artificial Sequence
  Synthetic Sequence
ggggccatggaacttcttcctcaagagtgga

SEQ ID NO: 29 - DNA, 31 bp, Artificial Sequence
  Synthetic Sequence
gggggtcgactcagtaaactgtaggcttaat

SEQ ID NO: 30 - DNA, 38 bp, Artificial Sequence
  Synthetic Sequence
ggggccatggatgaaaagaaaccctcacttcgtatcat

SEQ ID NO: 31 - DNA, 34 bp, Artificial Sequence
  Synthetic Sequence
gggggtcgacttatgattgcagagcaggagccat

SEQ ID NO: 32 - protein, 320 aa, Corynbacterium glutamicum
MTNIRVAIVGYGNLGRSVEKLIAKQPDMDLVGIFSRRATLDTKTPVFDVADVDKHADDVDVLFLCMGSATDIPEQAPKFAQFACTVDTYDNHRDIPRHRQVMNEAATAAGNVALVSTGWDPGMFSINRVYAAAVLAEHQQHTFWGPGLSQGHSDALRRIPGVQKAVQYTLPSEDALEKARRGEAGDLTGKQTHKRQCFVVADAADHERIENDIRTMPDYFVGYEVEVNFIDEATFDSEHTGMPHGGHVITTGDTGGFNHTVEYILKLDRNPDFTASSQIAFGRAAHRMKQQGQSGAFTVLEVAPYLLSPENLDDLIARDV

SEQ ID NO: 33 - protein, 326 aa, Bacillus Sphaericus
MSAIRVGIVGYGNLGRGVEFAISQNPDMELVAVFTRRDPSTVSVASNASVYLVDDAEKFQDDIDVMILCGGSATDLPEQGPHFAQWFNTIDSFDTHAKIPEFFDAVDAAAQKSGKVSVISVGWDPGLFSLNRVLGEAVLPVGTTYTFWGDGLSQGHSDAVRRIEGVKNAVQYTLPIKDAVERVRNGENPELTTREKHARECWVVLEEGADAPKVEQEIVTMPNYFDEYNTTVNFISEDEFNANHTGMPHGGFVIRSGESGANDKQILEFSLKLESNPNFTSSVLVAYARAAHRLSQAGEKGAKTVFDIPFGLLSPKSAAQLRKELL

SEQ ID NO: 34 - protein, 274 aa, Escherichia coli
MQQLQNIIETAFERRAEITPANADTVTREAVNQVIALLDSGALRVAEKIDGQWVTHQWLKKAVLLSFRINDNQVIEGAESRYFDKVPMKFADYDEARFQKEGFRVVPPAAVRQGAFIARNTVLMPSYVNIGAYVDEGTMVDTWATVGSCAQIGKNVHLSGGVGIGGVLEPLQANPTIIEDNCFIGARSEVVEGVIVEEGSVISMGVYIGQSTRIYDRETGEIHYGRVPAGSVVVSGNLPSKDGKYSLYCAVIVKKVDAKTRGKVGINELLRTID

SEQ ID NO: 35 - protein, 406 aa, Escherichia coli
MAIEQTAITRATFDEVILPIYAPAEFIPVKGQGSRIWDQQGKEYVDFAGGIAVTALGHCHPALVNALKTQGETLWHISNVFTNEPALRLGRKLIEATFAERVVFMNSGTEANETAFKLARHYACVRHSPFKTKIIAFHNAFHGRSLFTVSVGGQPKYSDGFGPKPADIIHVPFNDLHAVKAVMDDHTCAVVVEPIQGEGGVTAATPEFLQGLRELCDQHQALLVFDEVQCGMGRTGDLFAYMHYGVTPDILTSAKALGGGFPISAMLTTAEIASAFHPGSHGSTYGGNPLACAVAGAAFDIINTPEVLEGIQAKRQRFVDHLQKIDQQYDVFSDIRGMGLLIGAELKPQYKGRARDFLYAGAEAGVMVLNAGPDVMRFAPSLVVEDADIDEGMQRFAHAVAKVVGA

SEQ ID NO: 36 - protein, 375 aa, Escherichia coli
MSCPVIELTQQLIRRPSLSPDDAGCQALLIERLQAIGFTVERMDFADTQNFWAWRGQGETLAFAGHTDVVPPGDADRWINPPFEPTIRDGMLFGRGAADMKGSLAAMVVAAERFVAQHPNHTGRLAFLITSDEEASAHNGTVKVVEALMARNERLDYCLVGEPSSIEVVGDVVKNGRRGSLTCNLTIHGVQGHVAYPHLADNPVHRAAPFLNELVAIEWDQGNEFFPATSMQIANIQAGTGSNNVIPGELFVQFNFRFSTELTDEMIKAQVLALLEKHQLRYTVDWWLSGQPFLTARGKLVDAVVNAVEHYNEIKPQLLTTGGTSDGRFIARMGAQVVELGPVNATIHKINECVNAADLQLLARMYQRIMEQLVA

SEQ ID NO: 37 - protein, 273 aa, Bordetella parapertussis
MTLDLQTTIEQAWENRANLSPVDASAEVRDAVEHTIDGLDLGRLRVAEKIDDQWIVHQWIKKAVLLSFRLHDNAVMGQGPLQFYDKVPTKFAGYGEAAFKAGGYRVVPPAVARRGAFIARNVVLMPSYVNIGAYVDEGTMVDTWATVGSCAQIGKNVHLSGGVGIGGVLEPLQANPTIIEDNCFIGARSEVVEGVVVEENSVLAMGVFLSQSTKIYDRATGKVSYGRVPSGSVVVPGSLPSEDGSHSLTCAVIVKRVDAQTRAKTSINDLLRA

SEQ ID NO: 38 - protein, 435 aa, Bordetella parapertussis
MPGGRACARLQGFVALQSCGAPAARGLFPPLFLSPFRIPIMRKFSRIERLPPYVFNITGELKMAARRRGEDIIDMSMGNPDGATPQHIVDKLVEASTRPDTHGYSVSKGIPRLRKAICDWYMRRYAVEFDPDSEAIVTIGSKEGLAHLMLATLDRGDTVLVPNPSYPIHIYGAVIAGANIRSVRMTPGIDFFEELERAVRESIPKPKMMILGFPSNPTAQCVDLSFFERVVALAKEHDILVVHDLAYADVCFDGYVAPSIMQVPGARDVAVEFFTMSKSYNMAGWRIGYMVGNRELVGALARIKSYHDYGTFTPIQVASIAALDGPQDCVNEIVAQYQRRRDVLARGLHEAGWNVEIPKASMYIWAQIPEPYRAMGSLEFTKRVLSDAKVAVSPGIGFGEYGDEYVRFALIENEQRTRQAVRGIKDMFRKDGLLK

SEQ ID NO: 39 - protein, 379 aa, Bordetella parapertussis
MTASAVLDLVKDLIARPSVTPDDVDCQMLLAQRLERIGFQCETIARGGVTNLWARRGAGAPLTVFAGHTDVVPPGPRDKWDSDPFVPTERDGFLYGRGAADMKSSIAAFVVAAEEFVAAHPEHPGSIALLITSDEEGPAVDGTVIVCDELRQRGEQLDYCIVGEPTSTEALGDVCKNGRRGSLSGRLLVKGVQGHVAYPHLARNPVHQLAPALTELVAIEWDQGNEYFPPTTFQVSNLHAGTGATNVVPGEAVALFNFRFSTASTPDQLKARVHEVLDRHGLEYQLDWELGGEPFLTPRGSLTDALVSAIQAETGLQAELSTTGGTSDGRFIARICPQVIEFGPCNATIHKVNERIELSSLAPLKNIYRRTLENLLLAD